SEQUENCE LISTING (16 sequences)

SEQ ID NO: 1 - protein, 11 aa, Homo sapiens
  misc_feature (4)..(4): X1  is not A if X3 is N Otherwise X1 is any amino acid
TSFXEYWXLLX

SEQ ID NO: 2 - protein, 11 aa, Homo sapiens
  misc_feature (4)..(4): Xaa1 is any type of amino acid; and the peptide is a crosslinked peptide with a cross-linker to connect Xaa1 at position 4 to Xaa2 at position 11
TSFXEYWXLLX

SEQ ID NO: 3 - protein, 12 aa, Homo sapiens
  misc_feature (4)..(4): Xaa 1 at position 4 is any type of amino acid
TSFXEYWXLLXX

SEQ ID NO: 4 - protein, 11 aa, Homo sapiens
TSFAEYWNLLS

SEQ ID NO: 5 - protein, 11 aa, Homo sapiens
  Staple (4)..(4): X= staple position
TSFXEYWNLLX

SEQ ID NO: 6 - protein, 11 aa, Homo sapiens
TSFAEYWALLS

SEQ ID NO: 7 - protein, 11 aa, Homo sapiens
  misc_feature (4)..(4): Xaa can be any naturally occurring amino acid
TSFXEYWALLX

SEQ ID NO: 8 - protein, 11 aa, Homo sapiens
  staple (4)..(4): X= staple position; any amino acid
TSFXEYWALLX

SEQ ID NO: 9 - protein, 11 aa, Homo sapiens
  misc_feature (4)..(4): Xaa can be any naturally occurring amino acid
TSFXEYWALLX

SEQ ID NO: 10 - protein, 9 aa, Homo sapiens
  misc_feature (2)..(2): Xaa can be any naturally occurring amino acid
FXEYWALLX

SEQ ID NO: 11 - protein, 10 aa, Homo sapiens
  misc_feature (3)..(3): Xaa can be any naturally occurring amino acid
SFXEYWALLX

SEQ ID NO: 12 - protein, 11 aa, Homo sapiens
  misc_feature (4)..(4): Xaa can be any naturally occurring amino acid
TSFXEYWKLLX

SEQ ID NO: 13 - protein, 11 aa, Homo sapiens
  misc_feature (4)..(4): Xaa can be any naturally occurring amino acid
TSFXEYWYLLX

SEQ ID NO: 14 - protein, 11 aa, Homo sapiens
  misc_feature (4)..(4): Xaa can be any naturally occurring amino acid
TSFXEYWFLLX

SEQ ID NO: 15 - protein, 11 aa, Homo sapiens
  misc_feature (4)..(4): Xaa can be any naturally occurring amino acid
TSFXEYWILLX

SEQ ID NO: 16 - protein, 12 aa, Homo sapiens
ETFSDLWKLLPE

(19) United States
(12) Patent Application Publication
(10) Pub. No.: US 2015/0246946 A1
(43) Pub. Date: Sep. 3, 2015

(54) PEPTIDES AND METHODS FOR TREATING CANCER

(71) Applicant: AGENCY FOR SCIENCE, TECHNOLOGY AND RESEARCH, Singapore (SG)

(72) Inventors: Thomas Leonard Joseph, Singapore (SG); Chandra Shekhar Verma, Singapore (SG); David Phillip Lane, Singapore (SG); Christopher John Brown, Singapore (SG)

(21) Appl. No.: 14/432,729

(22) PCT Filed: Oct. 1, 2013
(86) PCT No.: PCT/SG2013/000428

§ 371 Date: Mar. 31, 2015

(30) Foreign Application Priority Data
Oct. 1, 2012 (SG) 201207302-9

(51) Int. Cl. C07K 7/06; A61K 38/08

(52) U.S. Cl. CPC C07K 7/06; A61K 38/08

(57) ABSTRACT

By using a phage display derived peptide as an initial template, compounds have been developed that are highly specific against Mdm2/Mdm4. These compounds exhibit greater potency in p53 activation and protein-protein interaction assays than a compound derived from the p53 wild-type sequence. Unlike nutlin, a small molecule inhibitor of Mdm2/Mdm4, the phage derived compounds can arrest cells resistant to p53 induced apoptosis over a wide concentration range without cellular toxicity, suggesting they are highly suitable for cyclotherapy.

This application claims the benefit of priority of SG provisional application No. 201207302-9, filed Oct. 1, 2012, the contents of it being hereby incorporated by reference in its entirety for all purposes.

### FIELD OF THE INVENTION

The present invention relates to the field of peptides and modified peptides for binding to Mdm2/Mdm4 and activating the p53 response.

### BACKGROUND OF THE INVENTION

Inhibition of the p53:Mdm2 interaction is an attractive therapeutic target. Molecules able to block the interaction can activate the p53 response by blocking the two inhibitory activities of Mdm2, namely its occlusion of the N-terminal p53 transactivation domain and its targeting of p53 for ubiquitination and proteasomal degradation. Such molecules could be used to re-activate p53 function in p53 wild-type tumour cells. In a second application, called cyclotherapy, their ability to induce a reversible cell cycle arrest in normal proliferating cells can selectively protect these tissues from cytotoxic chemotherapeutics and ionizing radiation, thus enabling the treatment of p53 null or p53 mutant tumours with fewer side effects.

Several classes of molecules that inhibit this interaction have been developed (e.g. Nutlin, MI-219). They mimic the conserved residues from a section of sequence in the p53 N-terminal that are essential for the interaction with the N-terminal p53 binding domain of Mdm2. This short sequence forms an α-helix upon binding, which allows the three conserved residues of the Mdm2 binding motif (FXXXWXXL) to optimally embed into the hydrophobic binding groove located on the surfaces of Mdm2 and the homologous Mdm4 proteins. However, these peptides exhibit off-target toxicity to p53-null cell and are not functional in the presence of serum.

Recent studies have reported a stapled peptide (SAH-8) derived from the wild type p53 sequence that binds to Mdm2 and Mdm4 and activates the p53 response in cells. However, the wild type p53 peptide (E¹TFSDLWKLLP¹¹E of SEQ ID No: 17) has a reported low affinity for Mdm2/Mdm4 (452±11 nM and 646±26 nM, respectively) and comes from a region of p53 that is known to interact with many other proteins.

Extensive studies have used phage display to select for linear peptides that bind Mdm2 with high affinity. However, the proline at position P12 is not observed in the electron density map in the crystal structure of the Mdm2:peptide complex and is not critical for binding to Mdm 2.

There is therefore a need to provide improved peptides or staple peptides that overcomes the disadvantages mentioned above to improve efficiency of binding to Mdm2/Mdm4 and reduce off target toxicity.

### SUMMARY OF THE INVENTION

According to a first aspect, there is provided a peptide comprising or consisting of the amino acid sequence of:

TSFXaa₁EYWXaa₃LLXaa₂

wherein Xaa₁ and Xaa₂ are any type of amino acid; and wherein Xaa₃ is N or A and wherein in case Xaa₃ is N, Xaa₁ is not A and/or Xaa₂ is not S.

According to a second aspect, there is provided a peptide comprising or consisting of the amino acid sequence of:

TSFXaa₁EYWXaa₃LLXaa₂

wherein Xaa₁, Xaa₂ and Xaa₃ is independently any type of amino acid; and wherein the peptide is a crosslinked peptide with a cross-linker to connect a first amino acid Xaa₁ to a second amino acid Xaa₂.

According to a third aspect, there is provided a peptide comprising or consisting of the amino acid sequence of

TSFXaa₁EYWXaa₃LLXaa₂Xaa₄

wherein Xaa₁, Xaa₂ and Xaa₃ is independently any type of amino acid; and wherein Xaa₄ is any type of amino acid other than P.

According to a fourth aspect, there is provided an isolated nucleic acid molecule encoding a peptide as described herein.

According to a fifth aspect, there is provided a vector comprising an isolated nucleic acid molecule as described herein.

According to a sixth aspect, there is provided a host cell comprising a nucleic acid molecule or a vector as described herein.

According to a seventh aspect, there is provided a pharmaceutical composition comprising a peptide as described herein, an isolated nucleic acid molecule as described herein, or a vector as described herein.

According to an eight aspect, there is provided the use of the peptide as described herein in the manufacture of a medicament for treating or preventing cancer.

According to a ninth aspect, there is provided a method of treating or preventing cancer in a patient comprising administering a pharmaceutically effective amount of a peptide as described herein or an isolated nucleic acid molecule as described herein or a vector as described herein.

### BRIEF DESCRIPTION OF THE DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is representative snapshot from simulations showing the crystal structure of SAH-8, MTide-01 and sMTide-01 in complex with Mdm2 and the biological activity of the peptides. (a) Crystal structure of SAH-8 in complex with Mdm2 (3V3B) showing the hydrocarbon staple interacting with the surface of the protein and the side-chain of T2 involved in helical capping hydrogen bond interactions with the backbone amide and side chain of N5. (b) A representative snapshot from a computer simulation of MTide-01 in complex with Mdm2 highlighting the hydrogen bond network around E5 and S2. S2 is involved in a weak hydrogen bond with the backbone amide of E5 and a much stronger hydrogen bond with the side chain of E5. In contrast to the wild type sequence E5 also interacts with the amide backbone of S2 further stabilizing the N-terminal of the bound helix resulting in optimal packing of the conserved F3 in the binding groove of Mdm2. (c) Overlay of representative snapshots from simulations of MTide-01 and sMTide-01 in complex with Mdm2 showing potential steric interference of N5 with the staple. The incorporation of the staple and the interactions it makes with the surface of Mdm2 causes the helix to roll. This orientates the staple into a position where it and the N5 side chain will modulate each other's fluctuations on the surface of Mdm2. (d) Linear and stapled peptides were tested for biological activity in a murine T22 cell line, which had been stably transfected with a p53 responsive LacZ reporter gene.

FIG. 2 shows titrations of Nutlin, sMTide-02, sMTide-02A, and SAH-8. (a) depicts line graphs of dose response curves showing titrations of Nutlin, sMTide-02, sMTide-02A, and SAH-8 in the T22 p53 transcriptional activity assay. (b) and (c) shows histograms representing titration data of stapled peptides, control peptides and Nutlin-3 into the F2H assay modelling the interaction of p53 with Mdm2 (b) or Mdm4 (c) in living BHK cells (from 0 to 50 μM). Graph bars show means of normalized interaction values (in %)±s.e.m. from three independent experiments. (d) shows images generated from a Western blot analysis of HCT-116 p53+/+ cells treated with either a 2-fold dilution series of SAH-8 or sMTide-02A peptide for 6 hours with or without fetal calf serum (FCS), respectively. (e) and (1) show the line graphs of titration data of nutlin and sMTide-02 into the T22 p53 reporter assay with and without 20 μM of the PGP efflux inhibitor PSC-883, respectively.

FIG. 3 is a series of histograms representing the biological activity of linear and stapled peptides in murine T22 cell line. Linear and stapled peptides were tested for biological activity in a murine T22 cell line, which was stably transfected with a p53 responsive LacZ reporter gene.

FIG. 4 is a series of histograms representing the toxicity and biological activity of peptides and stapled peptides. (a) shows histograms, indicating cell viability dose responses as indicated by intracellular ATP levels (Cell-Titer-Glow viability assay, Promega) of various cell lines against nutlin and the stapled peptides at 24 hours. (b) Caspase 3/7 activity dose responses (Caspase-Glo 3/7 assay, Promega) of various cell lines against nutlin and the stapled peptides at 24 hours. (c) shows flow cytometry histograms showing the cell cycle profiles of propidium iodide stained HCT116 p53+/+ cells in response to treatments with the stapled peptides and nutlin for 24 hours. (d) shows histograms representing Thymocytes from either wild-type or p53 knockout mice, which were isolated and treated with nutlin or sMTide-02/02A peptides. Thymocytes were stained with annexin V and PI and the percentage of viable cells (negative for PI and annexin V) after 24 hours were plotted against compound concentration.

FIG. 5 is flow cytometry chromatograms showing the effect of stapled peptides and nutlin on the cell cycle. FIG. 5 shows the cell cycle distribution of propidium iodide stained stained HCT116 p53+/+ cells in response to treatments with the stapled peptides and nutlin at 24 hours.

FIG. 6 is competitive fluorescence polarization titrations (represented as line graphs) demonstrating that the sMTide-02A/B peptides displace the FAM labeled probe from Mdm2 and Mdm4 respectively.

FIG. 7 is immunofluorescence imaging (micrographs) of F2H assay. Treatments of stapled peptides, control peptides and Nutlin-3 into the F2H assay modeling the interaction of p53 with Mdm2 (B) or Mdm4 (C) in live BHK cells at 5 μM for 6 hours. The F2H assay consists of two components, a bait and a prey proteins. The bait is a fusion of p53 with a lac repressor (LacI) and GFP, whilst the prey is a fusion of either Mdm2 or Mdm4 with RFP. Upon expression in the transgenic BHK cell line, the bait localizes to a distinct section of the chromosomal DNA containing stably integrated lac operator repeats and forms a bright green spot in the nucleus. The prey protein interacts with the bait protein and co-localizes at the same spot in the nucleus and forms a red spot. Compounds which inhibit the target interaction can then be titrated on to the cells and the declined percentage of co-localization can be measured using imaging techniques. The F2H assay differs from the p53 activity assay as it does not measure reactivation of a p53 reporter gene but the precise interaction to be disrupted.

### DETAILED DESCRIPTION OF THE PRESENT INVENTION

Before the present peptides, nucleic acids, vectors, pharmaceutical compositions, methods and uses thereof are described, it is to be understood that this invention is not limited to particular peptides, nucleic acids, vectors, pharmaceutical compositions, methods, uses and experimental conditions described, as such peptides, methods, uses and conditions may vary. It is also to be understood that the terminology used herein is for purposes of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

The invention is based, in part, on the discovery of peptides having improved pharmacological properties by using a phage display library. Advantageously, the peptide as disclosed herein is derived from a phage display library as an initial peptide, providing an experimental evidence of peptides which are potent binders against Mdm2/Mdm4, when compared to other inhibitors, such as inhibitors derived from the p53 wild-type sequences. The observations made by the inventors and disclosed herein are useful in the design of new Mdm2/Mdm4 inhibitors for therapeutic applications, for example, in the treatment of cancer.

Peptidomimetics represent an alternative approach to targeting eIF4E:eIF4G interaction. Proteins in their natural state are folded into regions of secondary structure, such as helices, sheets and turns. The alpha-helix is one of the most common structural motifs found in the proteins, and many biologically important protein interactions are mediated by the interaction of a α-helical region of one protein with another protein. Yet, α-helices have a propensity for unraveling and forming random coils, which are, in most cases, biologically less active, or even inactive, have lower affinity for their target, have decreased cellular uptake and are highly susceptible to proteolytic degradation.

Thus, the peptide as described herein exhibit a greater potency in p53 activation and protein-protein interaction assays than a compound derived from the p53 wild-type sequence. Exemplary, non-limiting embodiments of the peptides and cross-linked peptides for binding to Mdm2/Mdm4 and activating p53 response. In one embodiment, there is disclosed the peptide comprising or consisting of the amino acid sequence of SEQ ID NO 1 (TSFXaa₁EYW Xaa₃LLXaa₂), wherein Xaa₃ can be N or A and wherein in case Xaa₃ is N, Xaa₁ is not A and/or Xaa₂ is not S. In one embodiment, Xaa₃ can be any type of amino acid other than A and wherein in case Xaa₃ is N, Xaa₁ is not A and/or Xaa₂ is not S. Those peptides have been shown to inhibit the p53:Mdm2 interactions with a nanomolar affinity as illustrated in the experimental section (see e.g. example 1 and Table 1 below).

As defined herein, the terms “peptide”, “protein”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component. The term peptide encompasses two or more naturally occurring or synthetic amino acids linked by a covalent bond (e.g., a amide bond).

In the context of this specification, the term “amino acid” is defined as having at least one primary, secondary, tertiary or quaternary amino group, and at least one acid group, wherein the acid group may be a carboxylic, sulfonic, or phosphoric acid, or mixtures thereof. The amino, groups may be “alpha”, “beta”, “gamma” . . . to “omega” with respect to the acid group(s). Suitable amino acids include, without limitation, both the D- and L-isomers of the 20 common naturally occurring amino acids found in peptides (e.g., A, R, N, C, D, Q, E, G, H, I, L, K, M, F, P, S, T, W, Y, V (as known by the one letter abbreviations)) as well as the naturally occurring and unnaturally occurring amino acids prepared by organic synthesis or other metabolic routes.

The backbone of the “amino acid” may be substituted with one or more groups selected from halogen, hydroxy, guanido, heterocyclic groups. Thus term “amino acids” also includes within its scope glycine, alanine, valine, leucine, isoleucine, methionine, proline, phenylalanine, tryptophane, serine, threonine, cysteine, tyrosine, asparagine, glutamine, asparte, glutamine, lysine, arginine and histidine, taurine, betaine, N-methylalanine, etc. (L) and (D) forms of amino acids are included in the scope of this invention.

The term “amino acid side chain” refers to a moiety attached to the α-carbon in an amino acids. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an alpha di-substituted amino acid).

Thus, in one example, there is provided a peptide comprising or consisting of the amino acid sequence of SEQ ID NO: 2 (TSFXaa₁EYWXaa₃LLXaa₂), wherein Xaa₁, Xaa₂ and Xaa₃ is independently any type of amino acid and wherein the peptide is a crosslinked peptide with a cross-linker to connect a first amino acid Xaa₁ to a second amino acid Xaa₂. In another example, there is provided a peptide comprising or consisting of the amino acid sequence of SEQ ID NO: 3 (TSFXaa₁EYWXaa₃LLXaa₂Xaa₄), wherein Xaa₁, Xaa₂ and Xaa₃ is independently any type of amino acid and wherein Xaa₄ is any type of amino acid other than P. Accordingly, for example, Xaa₄ may comprise, but is not limited to, A, R, N, C, D, Q, E, G, H, I, L, K, M, F, S, T, W, Y, or V. In a further example, there is provided the peptide as described above, wherein Xaa₃ is N or A and wherein in case Xaa₃ is N, Xaa₁ is not A and/or Xaa₂ is not S.

As indicated above, peptides cross-linkers predominately increase the helicity of the peptide in solution before binding but this can be compromised by non-optimal interactions at the peptide:protein interface. In the rationally designed peptides of the invention such limitations have been overcome, or at least ameliorated by optimising packing effects at the interface, stabilising the bound complex and greater helical stabilization in solution. For example, in some peptides disclosed herein, the cross-linker might only induce 45% helicity but this can be compensated for with the formation of the (hydrogen) h-bond between two amino acids and by optimal packing interactions of another amino acid of the peptide. In contrast, another exemplary peptide may lose the hydrogen bond between the two amino acids upon binding but compensation arises via greater helicity (63%) in solution and stabilisation of the helical bound form by another amino acid. This is reflected in the enthalpy and entropy values of binding derived for these two peptides with the first exemplary peptide having a more favourable enthalpic component and the second exemplary peptide having a more favourable entropic component.

Many protein-protein interactions involve a contiguous section of protein that forms an interfacial α-helix when bound. Advantageously, this conformation can be further stabilized by a chemical method known as stapling, which consists of an all-hydrocarbon macrocyclic bridge connecting adjacent turns of the helix. Stapling peptides can increase their affinity by reducing the entropic cost of binding, increase their in vivo half-life by improving their proteolytic stability and most significantly allow their effective cellular uptake and intra-cellular activity. Thus, the present disclosure advantageously selected the peptides described above having a high affinity for Mdm2/Mdm4 to further improve their stability, protection from proteolytic cleavage and their cellular uptake, for example, by stapling.

The peptides may include at least one peptide cross-linker (also called a staple or a tether) between two non-natural (i.e. unnatural or synthetic) amino acids that significantly enhance the alpha helical structure of the peptides. Generally, the cross-linker extends across the length of one or two helical turns (that is about 3.4 or about 7 amino acids). Accordingly, amino acids positioned at i and i+3 (3 amino acids apart); and i and i+4; or i and i+7 are ideal candidates for chemical modification and cross-linking.

The term “cross-linker” or grammatical variations thereof as used herein refers to the intramolecular connection (also referred as staple) of two peptides domains (e.g., two loops of a helical peptide). When the peptide has a helical secondary structure, the cross-linker is a macrocyclic ring, which is exogenous (not part of) core or inherent (non-cross-linked) helical peptide structure. The macrocyclic ring may comprise an all-hydrocarbon linkage ring and incorporates the side chains linked to the α-carbon of at least two amino acids of the peptide. The size of the macrocyclic ring is determined by the number helical peptide amino acids in the ring and the number of carbon groups in the moieties connecting the α-carbon of the at least two amino acids of the peptide. The cross-linked peptide has at least one cross-linker. In various examples, the cross-linked peptide has 1, 2 or 3 cross linkers.

A cross-linked peptide (i.e. stapled peptide) is a peptide comprising a selected number of standard (i.e. natural) or non-standard (non-natural or unnatural or synthetic) amino acids, further comprising at least two moieties capable of undergoing reaction to promote carbon-carbon bond formation, that has been contacted with a reagent to generate at least one cross-link between the at least two moieties, which modulates, for example, peptide stability. The cross-linked peptide may comprise more than one, that is multiple (two, three, four, five, six, etc.) cross-links.

Any cross-linker known in the art can be used. Exemplary cross-linkers can include but are not limited to, hydrocarbon linkage, one or more of an ether, thioether, ester, amine, or amide moiety. In some cases, a naturally occurring amino acid side chain can be incorporated into the cross-linker. For example, a cross-linker can be coupled with a functional group such as the hydroxyl in serine, the thiol in cysteine, the primary amine in lysine, the acid in aspartate or glutamate, or the amide in asparagine or glutamine. Accordingly, it is possible to create a cross-link using naturally occurring amino acids rather than using a cross-linker that is made by coupling two non-naturally occurring amino acids. It is also possible to use a single non-naturally occurring amino acid together with a naturally occurring amino acid. In one example, there is provided a peptide as disclosed herein wherein the natural amino acid in the position to be cross-linked (i.e. the naturally occurring amino acid that is used to create the cross-linker) is replaced by an olefin-bearing unnatural amino acid. In a further example, the peptide as described above may comprise at least one two peptide cross linkers. Additionally, the peptide as described above is characterized by the presence of a first unnatural amino acid at the position Xaa₁ wherein the unnatural amino acid side chain cross-links to the side chain of a second unnatural amino acid at position Xaa₂. In one example, the cross-linker of the peptide as described herein may comprise a hydrocarbon linkage.

Thus, one example of a hydrocarbon linkage is the use of an olefin. The term “olefin” and grammatical variations thereof (also called alkene or alkenyl for a group) as used herein denotes a monovalent group derived from a straight- or branched-chain hydrocarbon moiety having at least one carbon-carbon double bond by the removal of a single hydrogen atom. The alkenyl moiety contains the indicated number of carbon atoms. For example, C₂-C₁₀ indicates that the group may have from 2 to 10 (inclusive) carbon atoms in it. The term “lower alkenyl” refers to a C₂-C₈ alkenyl chain. In the absence of any numerical designation, “alkenyl” is a chain (straight or branched) having 2 to 20 (inclusive) carbon atoms in it.

In certain embodiments, the olefinic group employed herein may contain 2-20 carbon atoms. In some embodiments, the olefin group employed herein may contain 2-15 carbon atoms. In another embodiment, the olefin group employed herein may contain 2-10 carbon atoms. In still other embodiments, the olefin group can contain 2-8 carbon atoms. In yet other embodiments, the olefinic group can contain 2-5 carbons, or 2, 3, 4, 5, 6, 7 or 8 carbons.

Olefinic groups include, for example, ethenyl, propenyl, butenyl, 1-methyl-2-buten-1-yl, and the like, which may bear one or more substituents. Olefinic group substituents include, but are not limited to, any of the substituents described herein, that result in the formation of a stable moiety. Examples of substituents include, but are not limited to, the following groups: aliphatic, alkyl, olefinic, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted. In one example, the olefinic groups are part of unnatural amino acids (R)-2-(7′octenyl) alanine and (S)-2-(4′-pentenyl) alanine. The side chains of the amino acids may stapled via olefin metathesis using the Grubbs catalyst.

In one embodiment, the peptide as disclosed herein may have a length of less than 11 amino acids; or between 4 to 15 amino acids; or between 4 to 11 amino acids; or between 6 to 11 amino acids; or between 8 to 11 amino acids. The length of the peptide may comprise, but is not limited to, 4, 5, 6, 7, 8, 9, 10 or 11 amino acids.

As used herein, the term “alkyl group” includes within its meaning monovalent (“alkyl”) and divalent (“alkylene”) straight chain or branched chain saturated aliphatic groups having from 1 to 10 carbon atoms, e.g., 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “alkenyl group” includes within its meaning monovalent (“alkenyl”) and divalent (“alkenylene”) straight or branched chain unsaturated aliphatic hydrocarbon groups having from 2 to 10 carbon atoms, e.g., 2, 3, 4, 5, 6, 7, 8, 9, or 10 carbon atoms and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl group” as used herein includes within its meaning monovalent (“alkynyl”) and divalent (“alkynylene”) straight or branched chain unsaturated aliphatic hydrocarbon groups having from 2 to 10 carbon atoms and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms, eg, 3, 4, 5, 6, 7, 8, 9, or 10 carbon atoms. Examples of cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like,

The term “heterocycloalkyl” as used herein, includes within its meaning monovalent (“heterocycloalkyl”) and divalent (“heterocycloalkylene”), saturated, monocyclic, bicyclic, polycyclic or fused hydrocarbon radicals having from 3 to 10 ring atoms wherein 1 to 5 ring atoms are heteroatoms selected from O, N, NH, or S. Examples include pyrrolidinyl, piperidinyl, quinuclidinyl, azetidinyl, morpholinyl, tetrahydrothiophenyl, tetrahydrofuranyl, tetrahydropyranyl, and the like.

The term “heteroaromatic group” and variants such as “heteroaryl” or “heteroarylene” as used herein, includes within its meaning monovalent (“heteroaryl”) and divalent (“heteroarylene”), single, polynuclear, conjugated and fused aromatic radicals having 6 to 20 atoms wherein 1 to 6 atoms are heteroatoms selected from O, N, NH and S. Examples of such groups include pyridyl, 2,2′-bipyridyl, phenanthrolinyl, quinolinyl, thiophenyl, and the like.

The term “halogen” or variants such as “halide” or “halo” as used herein refers to fluorine, chlorine, bromine and iodine.

The term “heteroatom” or variants such as “hetero-” as used herein refers to O, N, NH and S.

The term “alkoxy” as used herein refers to straight chain or branched alkyloxy groups. Examples include methoxy, ethoxy, n-propoxy, isopropoxy, tert-butoxy, and the like.

The term “amino” as used herein refers to groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

The term “aromatic group”, or variants such as “aryl” or “arylene” as used herein refers to monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like.

The term “aralkyl” as used herein, includes within its meaning monovalent (“aryl”) and divalent (“arylene”), single, polynuclear, conjugated and fused aromatic hydrocarbon radicals attached to divalent, saturated, straight and branched chain alkylene radicals.

The term “heteroaralkyl” as used herein, includes within its meaning monovalent (“heteroaryl”) and divalent (“heteroarylene”), single, polynuclear, conjugated and fused aromatic hydrocarbon radicals attached to divalent saturated, straight and branched chain alkylene radicals.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, isocyanate, —C(O)NH(alkyl), and —C(O)N(alkyl)₂.

The present invention includes within its scope all isomeric forms of the compounds disclosed herein, including all diastereomeric isomers, racemates and enantiomers. Thus, formulae (I) and (II) should be understood to include, for example, E, Z, cis, trans, (R), (S), (L), (D), (+), and/or (−) forms of the compounds, as appropriate in each case.

The term “substituted” is intended to indicate that one or more (e.g., 1, 2, 3, 4, or 5; in some embodiments 1, 2, or 3; and in other embodiments 1 or 2) hydrogen atoms on the group indicated in the expression using “substituted” is replaced with a selection from the indicated organic or inorganic group(s), or with a suitable organic or inorganic group known to those of skill in the art, provided that the indicated atom's normal valency is not exceeded, and that the substitution results in a stable compound. Suitable indicated organic or inorganic groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, alkylsilyl, and cyano. Additionally, the suitable indicated groups can include, e.g., —X, —R, —O—, —OR, —SR, —S—, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O—, —S(═O)₂OH, —S(═O)₂R, —OS(═O)₂OR, —S(═O)₂NR, —S(═O)R, —OP(═O)O₂ RR, —P(═O)O₂, RR—P(═O)(O—)₂, —P(═O)(OH)₂, —C(═O)R, —C(═O)X, —C(S)R, —C(O)OR, —C(O)O—, —C(S)OR, —C(O)SR, —C(S)SR, —C(O)NRR, —C(S)NRR, —C(NR)NRR, where each X is independently a halogen (or “halo” group): F, Cl, Br, or I; and each R is independently H, alkyl, aryl, heterocycle, protecting group or prodrug moiety. As would be readily understood by one skilled in the art, when a substituent is keto (i.e., ═O) or thioxo (i.e., ═S), or the like, then two hydrogen atoms on the substituted atom are replaced.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

In one example, there is provided the peptide as defined herein, wherein the peptide is a crosslinked peptide with a cross-linker to connect a first amino acid Xaa₁ to a second amino acid Xaa₂; and wherein Xaa₁, Xaa₂, Xaa₃ and Xaa₄ is independently any type of amino acid. For example Xaa₁, Xaa₂, Xaa₃ and Xaa₄ is independently A, R, N, C, D, Q, E, G, H, I, L, K, M, F, S, T, W, Y, or V in case the amino acid is a natural amino acid.

In one example, the peptide as disclosed herein may have a length of less than 11 amino acids; or between 4 to 15 amino acids; or between 4 to 11 amino acids; or between 6 to 11 amino acids; or between 8 to 11 amino acids. For example, the peptide as disclosed herein may a length of 4, 5, 6, 7, 8, 9, 10, or 11 amino acids.

In one example, the peptide as disclosed herein may be characterized in that the peptide inhibits the interaction between N-terminal region of p53 and Hdm2/Hdm4 and does not inhibit interaction between N-terminal region of p53 and other proteins which bind the N-terminal region of p53. As indicated in the examples below, the peptide as disclosed herein inhibits the interaction between N-terminal region of the p53 protein and the human Hdm2/Hdm4 protein. Advantageously, the binding of the peptide as disclosed herein interacts strongly and specifically with the human Mdm2/Mdm4 with low nanomolar K_{d}s.

In one example, the peptide as disclosed herein may have undergone a post-translational modification such as the addition of one, or two, or three, or four or more phosphoryl groups. In another example, the peptide may be modified to include one or more ligands comprising, but not limited to, hydroxyl, phosphate, amine, amide, sulphate, sulphide, a biotin moiety, a carbohydrate moiety, a fatty acid-derived acid group, a fluorescent moiety, a chromophore moiety, a radioisotope, a PEG linker, an affinity label, a targeting moiety, an antibody, a cell penetrating peptide or a combination of the aforementioned ligands. The addition of such ligands, may confer advantageous ability for the detection, tracking, activity, transport, pharmaceutical effect, or the stability of the peptide as disclosed herein.

In on example, the nitrogen of the backbone of the peptide as disclosed herein is methylated. In one example, the peptide as disclosed herein may be fused to a heterologous polypeptide sequence. The fusion of the peptide as disclosed herein to a polypeptide may allow forming a stapled protein larger than could practically be prepared using known peptide synthesis methodology. A synthetic peptide is ligated to a larger protein prepared recombinantly or purified from a natural source.

In one example, the tryptophan [W] at, for example position 7 of the peptide as disclosed, may be modified by addition of one or more halogen independently comprising F, Cl, Br, or I. This modification of the tryptophan of the peptide of interest may improve the potency of the peptide. In a further example, the tryptophan at position 7 of the peptide may comprise 1, 2, 3, 4, or 5 halogens comprising, but not limited to, F, Cl, Br, or I. For example, a tryptophan in the peptide may be modified by addition of a halogen at position C₁, C₂, C₃, C₄, C₅ or C₆ of W. In addition, W is independently an L or D optical isomer. In on example, the halogen is added at position C₆ of W. In a further example, the halogen may be Cl.

In one example, the peptide may comprise the formula:

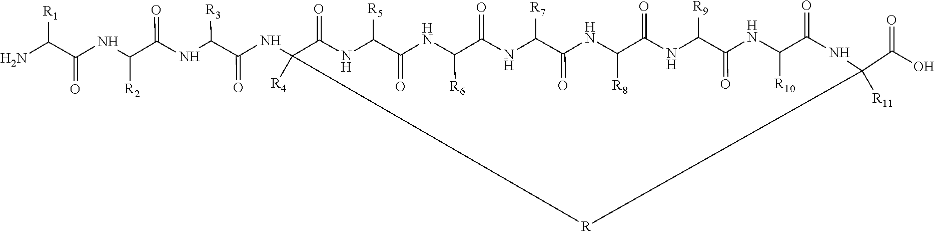

wherein R₁ is —C(OH)CH₃ [T]; R₂ is —CH₂OH [S]; R₃ is benzyl [F]; R₄ and R₁₁ are independently H or a C₁ to C₁₀ alkyl, alkenyl, alkynyl, arylalkyl, cycloalkylalkyl, heteroarylalkyl, or heterocyclylalkyl; R₅ is —(CH₂)₂C(O)OH [E]; R₆ is —CH₂-Phenyl-OH [Y]; R₇ is the side chain of Trp, wherein C₆ of Trp is substituted with a hydrogen or a halogen and/or wherein Trp is independently an L or D optical isomer; R₈ is the side chain of any amino acid; R₉ and R₁₀ are —CH₂CH(CH₃)₂ [L]; R is alkyl, alkenyl, alkynyl; [R′—K—R″]ₙ; each of which is substituted with 0-6 R₁₂; R′ and R″ are independently alkylene, alkenylene or alkynylene; each R₁₂ is independently halo, alkyl, OR₁₃, N(R₁₃)₂, SR₁₃, SOR₁₃, SO₂R₁₃, CO₂R₁₃, R₁₃, a fluorescent moiety, or a radioisotope; K is independently O, S, SO, SO₂, CO, CO₂, CONR₁₃ or; each R₁₃ is independently H, alkyl, or a therapeutic agent; n is an integer from 1-4. In the Experimental section (e.g. example 1) and Table 1 below, there is provided exemplary peptides, such as stapled peptides having the formula described above.

For example, sMTide-01 having the amino sequence of SEQ ID No: 5, (TSFX⁴EYWNLLX¹¹) and sMTide-02 having the amino acid sequence of SEQ ID No: 6 (TSFX⁴EYWNLLX¹¹) are such peptides. The X at position four is an unnatural amino acid (R)-2-(7′octenyl) alanine and the one at position eleven is (S)-2-(4′-pentenyl) alanine. The side chains were stapled by olefin metathesis using the Grubbs catalyst. Thus, in these examples, R₄ and R₁₁ are H and R is a C₁₁ alkenyl, resulting from the metathesis of an octenyl and a pentenyl.

In a further embodiment, R₄ and R₁₁ can be independently H or C₁-C₆ alkyl. In yet another embodiment, R is C₈ alkyl. In an embodiment, R can be C₁₁ alkyl. In an embodiment, R is alkenyl. In another embodiment, R can be a C₈ alkenyl. In yet another embodiment, R can be C₁₁ alkenyl.

In an embodiment, the peptide as described above may consist of R₁ is —C(OH)CH₃ [T], R₂ is —CH₂OH [S], R₃ is benzyl [F], R₅ is —(CH₂)₂C(O)OH [E], R₆ is —CH₂—Phenyl-OH [Y], R₇ is the side chain of Trp, wherein C₆ of Trp is substituted with a hydrogen or a halogen and/or wherein Trp is independently an L or D optical isomer, Re is the side chain of any amino acid, R₉ and R₁₀ are —CH₂CH(CH₃)₂ [L], [R′—K—R″]ₙ, each of which is substituted with 0-6 R₁₂, R₉ and R₁₀ are independently alkylene, alkenylene or alkynylene, each R₁₂ is independently halo, alkyl, OR₁₃, N(R₁₃)₂, SR₁₃, SOR₁₃, SO₂R₁₃, CO₂R₁₃, R₁₃, a fluorescent moiety, or a radioisotope, K is independently O, S, SO, SO₂, CO, CO₂, CONR₁₃ or each R₁₃ is independently H, alkyl, or a therapeutic agent, n is an integer from 1-4, R₄ and R₁₁ are H and R is C₁₁ alkenyl. In a further embodiment, R can be a linear chain alkyl, alkenyl or alkynyl.

In one embodiment, the peptide may comprise the formula:

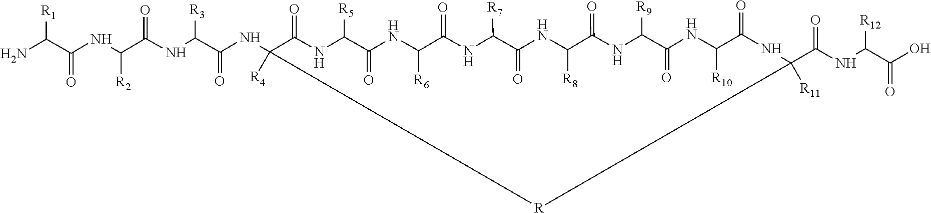

wherein R₁ is —C(OH)CH₃ [T]; R₂ is —CH₂OH [S]; R₃ is benzyl [F]; R₄ and R₁₁ are independently H or a C₁ to C₁₀ alkyl, alkenyl, alkynyl, arylalkyl, cycloalkylalkyl, heteroarylalkyl, or heterocyclylalkyl; R₅ is —(CH₂)₂C(O)OH [E]; R₆ is —CH₂-Phenyl-OH [Y]; R₇ is the side chain of Trp, wherein C₆ of Trp is substituted with a hydrogen or a halogen and/or wherein Trp is independently an L or D optical isomer, R₈ and R₁₂ are independently the side chain of any amino acid; R₉ and R₁₀ are —CH₂CH(CH₃)₂ [L]; R is alkyl, alkenyl, alkynyl; [R′—K—R″]ₙ; each of which is substituted with 0-6 R₁₂; R′ and R″ are independently alkylene, alkenylene or alkynylene; each R₁₃ is independently halo, alkyl, OR₁₄, N(R₁₄)₂, SR₁₄, SOR₁₄, SO₂R₁₄, CO₂R₄, R₁₄, a fluorescent moiety, or a radioisotope; K is independently O, S, SO, SO₂, CO, CO₂, CONR₁₄ or; each R₁₄ is independently H, alkyl, or a therapeutic agent; n is an integer from 1-4. In one embodiment, in the peptide described above, R₄ and R₁₁ can be independently H or C₁-C₆ alkyl. In one embodiment, R is C₈ alkyl. In one embodiment, R can be C₁₁ alkyl. In one embodiment, R can be alkenyl. In a further embodiment, R can be C₈ alkenyl. In yet another embodiment, R can be C₁₁ alkenyl.

In an embodiment, the peptide as described above may consist of R₁ is —C(OH)CH₃ [T], R₂ is —CH₂OH [S], R₃ is benzyl [F], R₅ is —(CH₂)₂C(O)OH [E], R₆ is —CH₂-Phenyl-OH [Y], R₇ is the side chain of Trp, wherein C₆ of Trp is substituted with a hydrogen or a halogen and/or wherein Trp is independently an L or D optical isomer, R₈ is the side chain of any amino acid, R₉ and R₁₀ are —CH₂CH(CH₃)₂ [L], R₇ is the side chain of Trp, wherein C₆ of Trp is substituted with a hydrogen or a halogen and/or wherein Trp is independently an L or D optical isomer, R₈ and R₁₂ are independently the side chain of any amino acid, R₉ and R₁₀ are —CH₂CH(CH₃)₂ [L], [R′—K—R″]ₙ, each of which is substituted with 0-6 R₁₂, R′ and R″ are independently alkylene, alkenylene or alkynylene, each R₁₃ is independently halo, alkyl, OR₁₄, N(R₁₄)₂, SR₁₄, SOR₄, SO₂R₁₄, CO₂R₁₄, R₁₄, a fluorescent moiety, or a radioisotope, K is independently O, S, SO, SO₂, CO, CO₂, CONR₁₄ or each R₁₄ is independently H, alkyl, or a therapeutic agent, n is an integer from 1-4, R₄ and R₁₁ are H; and R is C₁₁ alkenyl. Examples of alkyl, alkenyl or alkynyl are specifically disclosed herein.

In one embodiment, R can be a linear chain alkyl, alkenyl or alkynyl. Examples of possible are described in the art. In one example, there is provided the peptide as disclosed herein, wherein Xaa₃ is any type of amino acid other than A and wherein in case Xaa₃ is N, Xaa₁ is not A and/or Xaa₂ is not S. Xaa₃ may be for example, R, N, C, D, Q, E, G, H, I, L, K, M, F, S, T, W, Y, or V. In case Xaa₃ is N, Xaa₁ may comprise but is not limited to R, C, D, Q, E, G, H, I, L, K, M, F, S, T, W, Y, or V and/or Xaa₂ is not R, N, C, D, Q, E, G, H, I, L, K, M, F, T, W, Y, or V.

In some examples, there is provided an isolated nucleic acid molecule encoding a peptide as disclosed herein. In addition, the present invention also provides a nucleic acid molecule encoding for a peptide serving as template for the peptide of the present invention. Since the degeneracy of the genetic code permits substitutions of certain codons by other codons which specify the same amino acid and hence give rise to the same protein, the invention is not limited to a specific nucleic acid molecule but includes all nucleic acid molecules comprising a nucleotide sequence coding for the peptides of the present invention. The peptides encoded by the nucleic acid molecule may be chemically or enzymatically modified to obtain the cross-linked peptides as described herein.

The nucleic acid molecule disclosed herein may comprise a nucleotide sequence encoding the peptide serving as template for the peptide of the present invention which can be operably linked to a regulatory sequence to allow expression of the nucleic acid molecule. A nucleic acid molecule such as DNA is regarded to be ‘capable of expressing a nucleic acid molecule or a coding nucleotide sequence’ or capable ‘to allow expression of a nucleotide sequence’ if it contains regulatory nucleotide sequences which contain transcriptional and translational information and such sequences are “operably linked” to nucleotide sequences which encode the polypeptide. An operable linkage is a linkage in which the regulatory DNA sequences and the DNA sequences sought to be expressed are connected in such a way as to permit gene sequence expression. The precise nature of the regulatory regions needed for gene sequence expression may vary from organism to organism, but shall, in general include a promoter region which, in prokaryotes, contains only the promoter or both the promoter which directs the initiation of RNA transcription as well as the DNA sequences which, when transcribed into RNA will signal the initiation of synthesis. Such regions will normally include non-coding regions which are located 5′ and 3′ to the nucleotide sequence to be expressed and which are involved with initiation of transcription and translation such as the TATA box, capping sequence and CAAT sequences. These regions can for example, also contain enhancer sequences or translated signal and leader sequences for targeting the produced polypeptide to a specific compartment of a host cell, which is used for producing a peptide described above.

The nucleic acid molecule comprising the nucleotide sequence encoding the peptide as disclosed herein can be comprised in a vector, for example an expression vector. Such a vector can comprise, besides the above-mentioned regulatory sequences and a nucleic acid sequence which codes for a peptide as described above, a sequence coding for restriction cleavage site which adjoins the nucleic acid sequence coding for the peptide in 5′ and/or 3′ direction. This vector can also allow the introduction of another nucleic acid sequence coding for a protein to be expressed or a protein part. The expression vector preferably also contains replication sites and control sequences derived from a species compatible with the host that is used for expression. The expression vector can be based on plasmids well known to person skilled in the art such as pBR322, puC16, pBluescript and the like.

The vector containing the nucleic acid molecule can be transformed into host cells capable of expressing the genes. The transformation can be carried out in accordance with standard techniques. Thus, the invention is also directed to a (recombinant) host cell containing a nucleic acid molecule as defined above. In this context, the transformed host cells can be cultured under conditions suitable for expression of the nucleotide sequence encoding the peptide as described above. Host cells can be established, adapted and completely cultivated under serum free conditions, and optionally in media which are free of any protein/peptide of animal origin. Commercially available media such as RPMI-1640 (Sigma), Dulbecco's Modified Eagle's Medium (DMEM; Sigma), Minimal Essential Medium (MEM; Sigma), CHO-S-SFMII (Invitrogen), serum free-CHO Medium (Sigma), and protein-free CHO Medium (Sigma) are exemplary appropriate nutrient solutions. Any of the media may be supplemented as necessary with a variety of compounds, examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other-necessary supplements may also be included at appropriate concentrations that are known to those skilled in the art.

The peptide, the isolated nucleic acid molecule or the vector as described herein and above can be formulated into compositions, for example pharmaceutical compositions, suitable for administration. Where applicable, a peptide of the present invention may be administered with a pharmaceutically acceptable carrier. A “carrier” can include any pharmaceutically acceptable carrier as long as the carrier can is compatible with other ingredients of the formulation and not injurious to the patient. Accordingly, pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Therefore, the present invention also provides a pharmaceutical composition comprising a one or more peptide of the present invention.

A peptide as described above or pharmaceutical composition or medicament thereof can be administered in a number of ways depending upon whether local or systemic administration is desired and upon the area to be treated. In some embodiments, the peptide or the respective pharmaceutical composition thereof can be administered to the patient orally, or rectally, or transmucosally, or intestinally, or intramuscularly, or subcutaneously, or intramedullary, or intrathecally, or direct intraventricularly, or intravenously, or intravitreally, or intraperitoneally, or intranasally, or intraocularly.

The peptides themselves may be present in the compositions in any of a wide variety of forms. For example, two, three, four or more peptides may be merely mixed together or may be more closely associated through complexation, crystallization, or ionic or covalent bonding. The peptides of the invention can also encompass any pharmaceutically acceptable salts, esters, or salts of such esters, or any other compound, which, upon administration to an animal, including a human, is capable of providing the biologically active metabolite or residue thereof. Accordingly, also described herein is drawn to prodrugs and pharmaceutically acceptable salts of such pro-drugs, and other bioequivalents. The term “pharmaceutically acceptable salt” refers to physiologically and pharmaceutically acceptable salt(s) of the peptides as described above; i.e. salts that retain the desired biological activity of the peptide and do not impart undesired toxicological effects thereto. Examples of such pharmaceutically acceptable salts include but are not limited to (a) salts formed with cations such as sodium, potassium, ammonium, magnesium, calcium, polyamines such as spermine and spermidine, etc; (b) acid addition salts formed with inorganic acids, for example hydrochloric acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chorine, bromine, and iodine.

In some embodiments, the pharmaceutical composition as described above and herein may further comprise at least one, or at least two, or at least three or more therapeutic compound (or an agent or a molecule or a composition). A “therapeutic” compound as defined herein is a compound (or an agent or a molecule or a composition) capable of acting prophylactically to prevent the development of a weakened and/or unhealthy state; and/or providing a subject with a sufficient amount of the complex or pharmaceutical composition or medicament thereof so as to alleviate or eliminate a disease state and/or the symptoms of a disease state, and a weakened and/or unhealthy state.

In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

In the context of this specification the terms “therapeutically effective amount” and “diagnostically effective amount”, include within their meaning a sufficient but non-toxic amount of a compound or composition of the invention to provide the desired therapeutic or diagnostic effect. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

In one example, the therapeutic compound includes but is not limited to an apoptosis promoting compound, a chemotherapeutic compound or a compound capable of alleviating or eliminating cancer in a patient. Examples of apoptosis promoting compounds include but are not limited to Cyclin-dependent Kinase (CDK) inhibitors, Receptor Tyrosine Kinase (RTK) inhibitors, BCL (B-cell lymphoma) family BH3 (Bcl-2 homology domain 3)-mimetic inhibitors and Ataxia Telangiectasia Mutated (ATM) inhibitors.

In an example, the Cyclin-dependent Kinase (CDK) inhibitors include but are not limited to 2-(R)-(1-Ethyl-2-hydroxyethylamino)-6-benzylamino-9-isopropylpurine (CYC202; Roscovitine; Seliciclib); 4-[[5-Amino-1-(2,6-difluorobenzoyl)-1H-1,2,4-triazol-3-yl]amino]benzenesulfonamide (JNJ-7706621); N-(4-piperidinyl)-4-(2,6-dichlorobenzoylamino)-1H-pyrazole-3-carboxamide (AT-7519); N-(5-(((5-(1,1-dimethylethyl)-2-oxazolyl)methyl)thio)-2-thiazolyl)-4-piperidinecarboxamide (SNS-032); 8,12-Epoxy-1H,8H-2,7b,12a-triazadibenzo(a,g)cyclonona(cde)triinden-1-one, 2,3,9,10,11,12-hexahydro-3-hydroxy-9-methoxy-8-methyl-10-(methylamino)-(UCN-01; 7-Hydroxystaurosporine; KRX-0601); N, 1,4,4-tetramethyl-8-((4-(4-methylpiperazin-1-yl)phenyl)amino)-4,5-dihydro-1H-pyrazolo[4,3-h]quinazoline-3-carboxamide (PHA-848125; milciclib); 2-(2-chlorophenyl)-5,7-dihydroxy-8-[(3S,4R)-3-hydroxy-1-methylpiperidin-4-yl]chromen-4-one hydrochloride (flavopiridol; alvocidib); 6-acetyl-8-cyclopentyl-5-methyl-2-((5-(piperazin-1-yl)pyridin-2-yl)amino)pyrido[2,3-d]pyrimidin-7(8H)-one hydrochloride (PD 0332991); 4-(1-isopropyl-2-methyl-1H-imidazol-5-yl)-N-(4-(methylsulfonyl)phenyl)pyrimidin-2-amine (AZD5438); (S)-3-(((3-ethyl-5-(2-(2-hydroxyethyl)piperidin-1-yl)pyrazolo[1,5-a]pyrimidin-7-yl)amino)methyl)pyridine 1-oxide (Dinaciclib; SCH 727965); N-(4-Piperidinyl)-4-(2,6-dichlorobenzoylamino)-1H-pyrazole-3-carboxamide hydrochloride (AT-7519); and pharmaceutically acceptable salts thereof.

In another example, there is provided the pharmaceutical composition as described above, wherein the RTK inhibitors include but are not limited to N-[3-chloro-4-[(3-fluorophenyl)methoxy]phenyl]-6-[5-[(2-methylsulfonylethylamino)methyl]-2-furyl]quinazolin-4-amine (lapatinib); N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (foretinib); N-(4-((6,7-Dimethoxyquinolin-4-yl)oxy)phenyl)-N-(4-fluorophenyl)cyclopropane-1,1-dicarboxamide (cabozantinib (XL184)); N-(4-((6,7-Dimethoxyquinolin-4-yl)oxy)phenyl)-N-(4-fluorophenyl)cyclopropane-1,1-dicarboxamide (cabozantinib (XL184)); 3-[(1R)-1-(2,6-dichloro-3-fluorophenyl)ethoxy]-5-(1-piperidin-4-ylpyrazol-4-yl)pyridin-2-amine (crizotinib (Xalkori)); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxo-2,3-dihydro-1H-indole-5-sulfonamide (SU11274); (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one hydrate (PHA-665752); 6-[[6-(1-Methylpyrazol-4-yl)-[1,2,4]triazolo[4,3-b]pyridazin-3-yl]sulfanyl]quinoline (SGX-523); 4-[1-(6-Quinolinylmethyl)-1H-1,2,3-triazolo[4,5-b]pyrazin-6-yl]-1H-pyrazole-1-ethanol methanesulfonate (1:1) (PF-04217903); 2-Fluoro-N-methyl-4-[7-[(quinolin-6-yl)methyl]imidazo[1,2-b]-[1,2,4]triazin-2-yl]benzamide (INCB28060); N-[4-[(3-Chloro-4-fluorophenyl)amino]-7-[[(3S)-tetrahydro-3-furanyl]oxy]-6-quinazolinyl]-4(dimethylamino)-2-butenamide (afatinib); 3-(5,6-Dihydro-4H-pyrrolo[3,2,1-ij]quinolin-1-yl)-4-(1H-indol-3-yl)-pyrrolidine-2,5-dione (ARQ-197 (Tivantinib)); N-[(2R)-1,4-dioxan-2-ylmethyl]-N-methyl-N-[3-(1-methyl-1H-pyrazol-4-yl)-5-oxo-5H-benzo[4,5]cyclohepta[1,2-b]pyridin-7-yl]sulfuric diamide (MK-2461); N-[4-(3-Amino-1H-indazol-4-yl)phenyl]-N-(2-fluoro-5-methylphenyl)urea (Linifanib (ABT 869));

4-[[(3S)-3-Dimethylaminopyrrolidin-1-yl]methyl]-N-[4-methyl-3-[(4-pyrimidin-5-ylpyrimidin-2-yl)amino]phenyl]-3-(trifluoromethyl)benzamide (Bafetinib (INNO-406)); and pharmaceutical acceptable salts thereof.

In a further example, there is provided the pharmaceutical composition as described above, wherein the BCL family BH3-mimetic inhibitors include but are not limited to: 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (ABT 263; Navitoclax);

2-[2-[(3,5-Dimethyl-1H-pyrrol-2-yl)methylene]-3-methoxy-2H-pyrrol-5-yl]-1H-indole methanesulfonate (Obatoclax mesylate (GX15-070)); 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737); and pharmaceutically acceptable salts thereof.

In an additional example, there is provided the pharmaceutical composition as described above, wherein the ATM inhibitors comprise inhibitors include but are not limited: 2-Morpholin-4-yl-6-thianthren-1-yl-pyran-4-one (KU-55933); (2R,6S)-2,6-Dimethyl-N-[5-[6-(4-morpholinyl)-4-oxo-4H-pyran-2-yl]-9H-thioxanthen-2-yl]-4-morpholineacetamide (KU-60019); 1-(6,7-Dimethoxy-4-quinazolinyl)-3-(2-pyridinyl)-1H-1,2,4-triazol-5-amine (CP466722); α-Phenyl-N-[2,2,2-trichloro-1-[[[(4-fluoro-3-nitrophenyl)amino]thioxomethyl]amino]ethyl]benzene acetamide (CGK 733) and pharmaceutically acceptable salts thereof.

In some embodiments, the cancer treated or prevented in the invention may be any form of a cancer. Any forms of tumor or cancer may be used in the invention including for example, a benign tumor and a metastatic malignant tumor. Examples of cancers include, but are not limited to, gastric cancer, colon cancer, lung cancer, breast cancer, bladder cancer, neuroblastoma, melanoma, head and neck cancer, esophagus cancer, cervix cancer, prostate cancer and leukemia.

In one embodiment, the administration of the peptide may induce a reversible cell cycle arrest in non-cancerous proliferating cells. The present invention also provides the use of a peptide as described herein in the manufacture of a medicament for treating or preventing cancer. In another embodiment, the patient suffering or suspected to suffering from cancer may comprises a tumor with p53 deficient tumor cells or p53 genes comprising a mutation which causes the cancer. In some embodiments, the cancer as described above is characterized by low expression or inhibition of p53 containing complexes. As used herein, the term “low expression” denotes a level of expression of the proteins in a complex that comprises p53 that is below a level found in cells isolated or cultivated from a patient having no disease or being healthy. For example, inhibition of p53 may be found in the cancer cells isolated from a cancer patient as compared to the expression level of p53 in the non-cancer cells of the patient or in the cells isolated from an healthy patients, wherein the cells belong to the same group having the same histological, morphological, physical, and biological characteristics (e.g. hepatocytes, keratinocytes, lung cells . . . ). As used herein the term “inhibition” denotes a level of enzymatic, biological, dynamic or any measurable activity of the proteins in a complex that comprises p53 that is below a level found in cells isolated or cultivated from a healthy patient having no diseases, conditions or any ailments. For example, inhibition of p53 may be found in the cancer cells isolated from a cancer patient as compared to the activity level of p53 protein in the non-cancer cells of the patient or in the cells isolated from a healthy patients, wherein the cells belong to the same group having the same histological, morphological, physical, and biological characteristics.

In one example, there is provided the use of the peptide as described herein in the manufacture of a medicament for treating or preventing cancer. In another example, there is provided the use of the peptide as described above, wherein the cancer comprises a tumor comprising a non-mutant p53 sequence. In other words, the peptide may be used to activate p53 or inhibit its degradation in a cancer in which the p53 protein is a wild type p53 protein. The peptide as disclosed herein may be used to inhibit the p53:Mdm2 interaction. Accordingly, the peptide is useful in the manufacture of a medicament to block the interaction, which in turn can activate the p53 response by blocking the two inhibitory activities of Mdm2, namely its occlusion of the N-terminal p53 transactivation domain and its targeting of p53 for ubiquitination and proteasomal degradation. The peptide as disclosed herein can thus be used to re-activate p53 function in p53 wild-type tumour cells.

Accordingly, the use described above may be used in cancer comprising, but not limited to, gastric cancer, colon cancer, lung cancer, breast cancer, bladder cancer, neuroblastoma, melanoma, or leukemia. Other examples of tumors include, but are not limited to, haematological malignancies and solid tumours. Solid tumours include for instance a sarcoma, arising from connective or supporting tissues, a carcinoma, arising from the body's glandular cells and epithelial cells or a lymphoma, a cancer of lymphatic tissue, such as the lymph nodes, spleen, and thymus.

In another example, there is provided a method of treating or preventing cancer in a patient comprising administering a pharmaceutically effective amount of the peptide as disclosed herein or the isolated nucleic acid molecule as disclosed herein, or the vector as disclosed herein. The administration of either a peptide, protein fused to the peptide, a nucleic acid encoding the peptide or the vector comprising said nucleic as described above may be achieved by different means as described herein and will result in the expression of the inhibitor of the p53:Mdm2 interactions, thereby to the reactivation of p53 function.

The term “treat” or “treating” as used herein is intended to refer to providing an pharmaceutically effective amount of a peptide of the present invention or a respective pharmaceutical composition or medicament thereof; sufficient to act prophylactically to prevent the development of a weakened and/or unhealthy state; and/or providing a subject with a sufficient amount of the complex or pharmaceutical composition or medicament thereof so as to alleviate or eliminate a disease state and/or the symptoms of a disease state, and a weakened and/or unhealthy state.

In the context of this invention the term “administering” and variations of that term including “administer” and “administration”, includes contacting, applying, delivering or providing a compound or composition of the invention to an organism, or a surface by any appropriate means.

Thus, in some embodiments there is provided a method of treating or preventing cancer in a patient in need thereof. The method includes administering of a pharmaceutically effective amount of a peptide, the isolated nucleic acid or the vector as described above and herein. The method of the invention can in some embodiments include administering the pharmaceutically effective amount of the peptide with one or more further therapeutic compounds, wherein administration is simultaneous, sequential or separate.

### EXPERIMENTAL SECTION

Non-limiting examples of the invention and comparative examples will be further described in greater detail by reference to specific Examples, which should not be construed as in any way limiting the scope of the invention.

### Example 1

This example demonstrates the selection of the most suitable peptide for binding to Mdm2/Mdm4 by a combining phage display and computational techniques. The currently most avid published peptides were used as the template for this study. One of these peptides, termed MTide-01 (T¹SFAEYWNLLS¹¹; having the amino acid SEQ ID NO: 4), interacts strongly and specifically with Mdm2/Mdm4 with low nanomolar K_{d}s (Table 1).

TABLE 1
Apparent K_{d}s were determined by competitive fluorescence polarization.
Presence of I, I + 7 macrocyclic hydrocarbon bridge indicated by X.

| | | Mdm2 K_{d} | Mdm4 K_{d} | SEQ ID |
|---|---|---|---|---|
| Ligand | Primary Sequence | (nM) | (nM) | NO: |
| MTide-01 | Ac-¹TSFAEYWNLLS¹¹-NH2 | 46.34 ± 6.89 | 33.16 ± 4.62 | 4 |
| sMTide-01 | Ac-¹TSFXEYWNLLX¹¹-NH2 | 86.99 ± 0.02 | 118.3 ± 0.04 | 5 |
| MTide-02 | Ac-¹TSFAEYWALLS¹¹-NH2 | 28.04 ± 1.38 | 16.33 ± 2.00 | 6 |
| sMTide-02 | Ac-¹TSFXEYWALLX¹¹-NH2 | 34.35 ± 2.03 | 45.73 ± 7.65 | 7 |
| sMTide-02A | Ac-¹TSFXEY(L-6-Cl)WALLX¹¹-NH2 | 6.76 ± 2.11 | 1360 ± 600 | 8 |
| sMTide-02B | Ac-¹TSFXEY(D-6-Cl)WALLX¹¹-NH2 | 88.16 ± 7.20 | 2160.73 ± 1000 | 9 |
| SAH-8 | Ac-QSQ¹QTFXNLWRLLX¹¹QN-NH2 | 126.09 ±13.59 | 14.03 ± 1.85 | 10 |

The staple was incorporated across positions 4 and 11 in MTide-01 to create the stapled derivative sMTide-01, having the amino acid SEQ ID No: 5. The Proline at position P¹² was removed from the original sequence, as the computer simulations demonstrated that induction of the helix by the staple would prevent the proline from packing optimally against the Mdm4 surface. Also P12 is not observed in the electron density map in the crystal structure of the Mdm2:peptide complex and is not critical for binding to Mdm2.

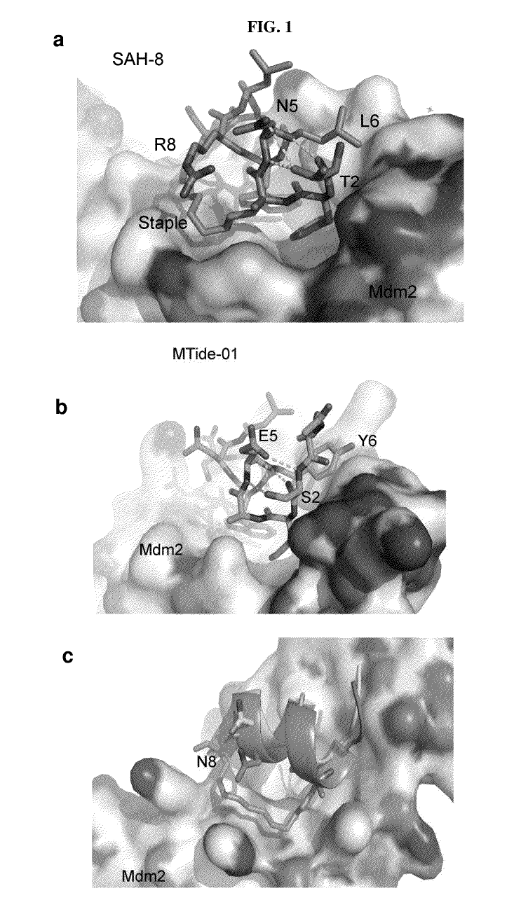

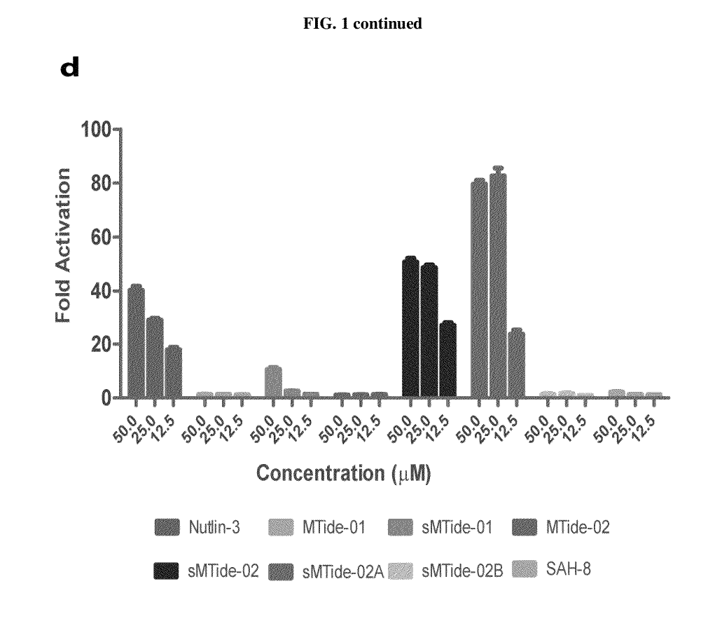

The mechanism responsible for the improved binding of MTide-01 in comparison to the wild type sequence was supported by simulation data. The mechanism is the optimization of an intramolecular hydrogen bond network that stabilizes its helical conformation when bound to Mdm2/Mdm4, which is principally centered on S2 and E5 (FIG. 1b). Y6 makes extensive van der Waals contacts with Mdm2/Mdm4 and also participates in a hydrogen bond network on the surface of Mdm2/4 via its hydroxyl group. The replacement of P11 with S11 allows the c-terminal of the peptide to adopt a helical structure, increasing the helicity of the peptide. This position also corresponds to where the staple is tethered in sMTide-01, thereby replacing the helical inducing property of S11. Interestingly, alanine scanning mutagenesis identified N8 as being detrimental to Mdm2/4 binding primarily through disruption of the bound helix as Asn is rarely located within solvent exposed central regions of α-helices. Asn 8 was also identified computationally to interfere sterically with the putative placement of the staple and as a result was replaced with the favourable Ala mutation to create the sequence termed MTide-02 and the stapled derivative sMTide-02 (FIG. 1c).

### Example 2

This example provides a comparison of the affinity of the peptides of the present invention and previously disclosed peptides for Mdm2/Mdm4. sMTide-02 yielded the most potent derivative with a K_{d} of 34.60±2.03 nM against Mdm2 and a K_{d} of 45.73±7.65 nM against Mdm4 (Table 1). In comparison to their unstapled counterparts, both peptides exhibited slightly weaker K_{d}s with sMTide-01 showing an approximately 2-fold increase against Mdm2 and a 4-fold increase against Mdm4. In contrast, sMTide-02 had a negligible increase against Mdm2 whilst showing a 2-fold increase in K_{d} against Mdm4. These results validate the hypothesis that N8 interferes with the staple and the stabilization of the helix within sMTide-01. Both peptides were then tested for biological activity (FIG. 1 (d)). sMTide-01 displayed little activity in a T22 derived p53 reporter cell line. Surprisingly, it was discovered that sMTide-02 induced the strongest p53 transcriptional response ever seen in this assay, which has been used to screen more than 300,000 compounds.

The SAH-8 peptide was synthesized and its K_{d}s were determined to be 126.09±13.59 nM and 14.03±1.85 nM against Mdm2 and Mdm4 respectively. In contrast to sMTide-01/02, it showed a significant preference for Mdm4. When tested in the T22 p53 reporter assay, SAH-8 had negligible activity compared to sMTide-01 and induced considerably lower levels of p53 than sMTide-02 (FIG. 1d). Interestingly, the placement of the staple in the SAH-8 sequence caused a considerable increase in affinity of the peptide against Mdm2 and Mdm4, in contrast to the MTide based sequences (see Table 1).

The origin of this improvement in affinity for Mdm2/4 lay in the sequence used to design SAH-8, which had not been optimized to stabilize the helical form of the peptide. The staple of SAH-8 more than compensated for this by reducing the entropic cost of binding and by making additional hydrophobic contacts with the surface of Mdm2/4. However, incorporation of the I, I+7 staple into the MTide-01 and 02 sequences lead to no additional increase in affinity for Mdm2/4. This highlighted the structural and energetic intricacies of adding a staple to a peptide with pre-existing helical stabilizing interactions, and their associated effects on the affinity of the peptide for the protein.

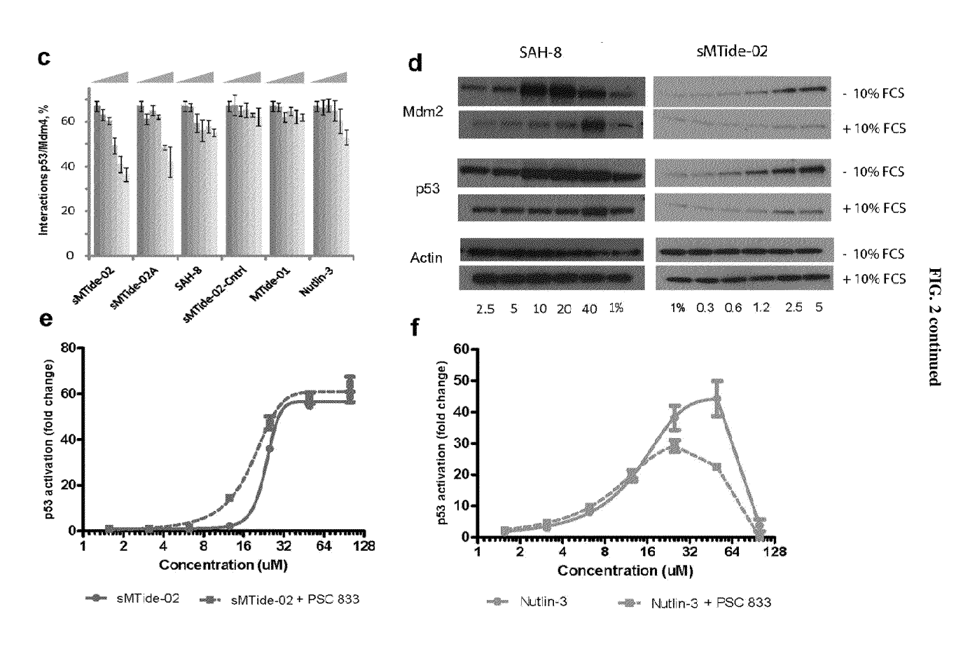

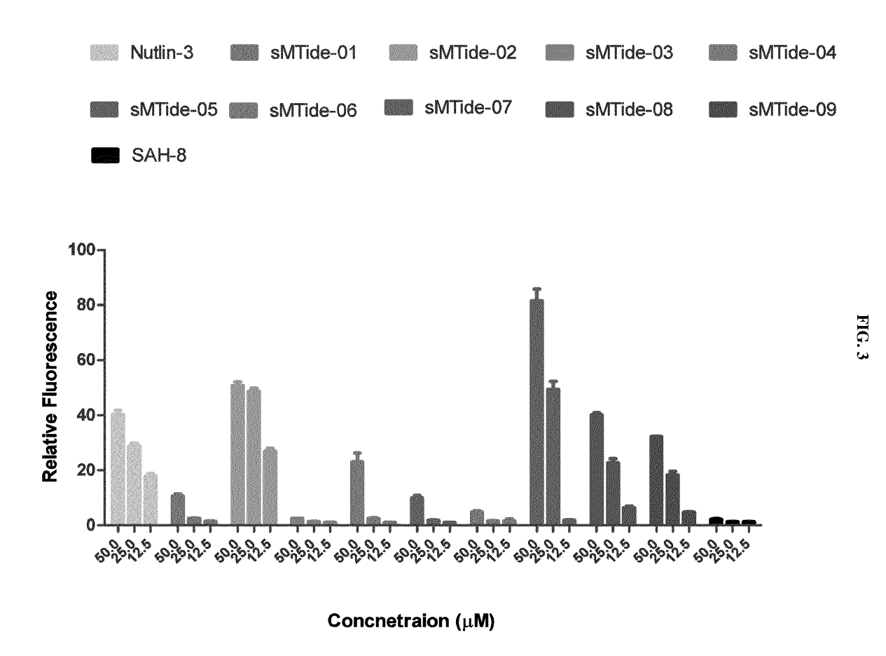

Other analogs of sMTide-02 were designed either by truncating the N-terminal of the stapled peptide or by introducing alternative substitutions at position 8. Truncation of sMTIDE-02 at the N-terminal attenuated the affinity of the peptide for Mdm2/4 with concomitant decreases in their activity in the T22 p53 reporter assay (FIG. 3 and Table 2). Interestingly, sMTide-04, where threonine at position 1 has been removed, interacted more tightly with Mdm4 than with Mdm2. Substitution of alanine at position 8 with a variety of amino acids generated a set of stapled peptides (FIG. 3 and Table 2) with a wide range of K_{d}s that did not correlate with activity in the p53 reporter assay.

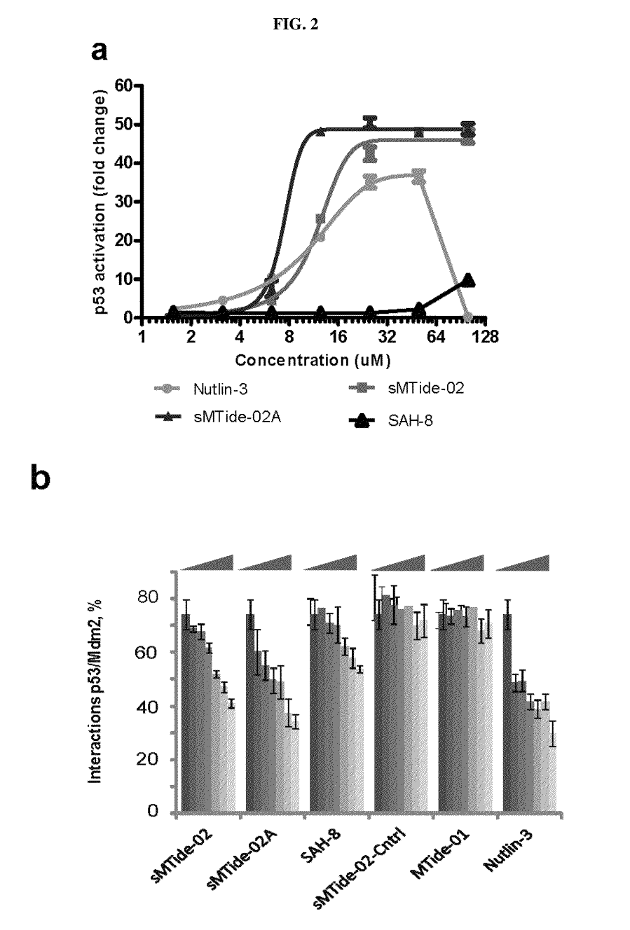

For example, if isoleucine and phenylalanine were introduced at position 8, the binding of the stapled peptide to Mdm2/4 was dramatically attenuated compared to sMTide-01, but their biological activity in the p53 reporter assay was improved. These results show that the context of the sequence, within which the staple is utilized, ultimately determines its effectiveness and that there is a complex relationship between the affinity of the compound for its target protein and its biological activity. Intriguingly, the small addition of a hydroxyl, i.e. phenylalanine vs. tyrosine, (sMTide-07 and 06, respectively) had little effect on the interaction between these two peptides and Mdm2/4. However, it did diminish the ability of sMTide-07 to induce p53, compared to sMTide-06. Presumably, these types of changes influenced the ability of the stapled peptide to enter cells efficiently, either by limiting cell permeability or perhaps by causing endosomal entrapment. SAH-8, on the other hand, which interacted much more tightly than both of these molecules with Mdm2/4, was a very poor activator of p53 activity in the T22 assay (FIG. 1 (d) and FIG. 2 (a)). However, SAH-8 possesses a sequence that differs significantly from sMTide-02 and as a result this makes it harder to delineate the precise differences for its poor biological activity in physiochemical terms.

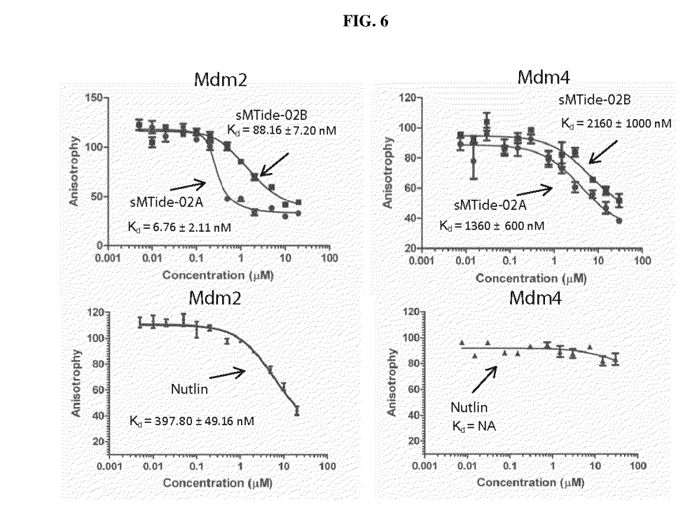

A modification that is known to improve the potency of peptides that interact with Mdm2/4 is the addition of a Chlorine atom at the C6 position of W7. Two stapled peptide analogues of sMTide-02 were synthesized containing either the L (termed sMTide-02A) or D 0.30 (termed sMTide-02B) optical isomers of the 6-Cl modified tryptophan. The L-isomer bound Mdm2 with an improved K_{d} of 6.76±2.11 nM, but its affinity for Mdm4 was significantly attenuated; this preference for Mdm2 over Mdm4 is also seen for nutlin (Table 1 and FIG. 6). The D-isomer showed negligible activity in the T22 assay, despite interacting with Mdm2 with an apparent K_{d} of 88.16 nM±7.20, indicating either poor cell permeability or an inability to disrupt the pre-existing p53:Mdm2 interactions within the cell. As with the L optical isomer, its K_{d} with Mdm4 was attenuated (Table 1). The L isomer sMTide-02A showed a much higher fold induction of p53 transcriptional activity than the unmodified sMTide-02 in the T22 assay (FIG. 1 (d)).

TABLE 2
Apparent K_{d}s were determined by competitive fluorescence polarization.
Presence of I, I + 7 macrocyclic hydrocarbon bridge indicated by X.

| | | Mdm2 K_{d} | Mdm4 K_{d} | SEQ ID |
|---|---|---|---|---|
| Ligand | Primary Sequence | (nM) | (nM) | No: |
| sMTide-03 | Ac-³FXEYWALLX¹¹-NH2 | N/A | N/A | 11 |
| sMTide-04 | Ac-²SFXEYWALLX¹¹-NH2 | 103.7 ± 0.1 | 63.0 ± 0.1 | 12 |
| sMTide-05 | Ac-¹TSFXEYWKLLX¹¹-NH2 | 26.3 ± 0.1 | 29.9 ± 0.1 | 13 |
| sMTide-06 | Ac-¹TSFXEYWYLLX¹¹-NH2 | 127.7 ± 0.2 | 328.5 ± 0.2 | 14 |
| sMTide-07 | Ac-¹TSFXEYWFLLX¹¹-NH2 | 174.6 ± 31.10 | 394.30 ± 102.20 | 15 |
| sMTide-08 | Ac-¹TSFXEYWILLX¹¹-NH2 | 127.1 ± 18.56 | 205.70 ± 54.89 | 16 |

### Example 3

This example provides a titration of p53 activating peptides. Titrations of p53 activating compounds into the T22 assay typically produced a bell shaped curve in which high concentrations of compound produced lower levels of reporter protein as a result of cell toxicity. Remarkably, while nutlin induced a typical bell shaped curve, the two peptides showed a sigmoidal curve with a plateau over a large dose range, with much higher levels of reporter protein production, indicating that these compounds lack cell toxicity despite their ability to activate p53 function to high levels (FIG. 2a). However, both stapled peptides at low concentrations induced less p53 activity than nutlin, indicating that the dynamics of cell entry for these molecules are different.

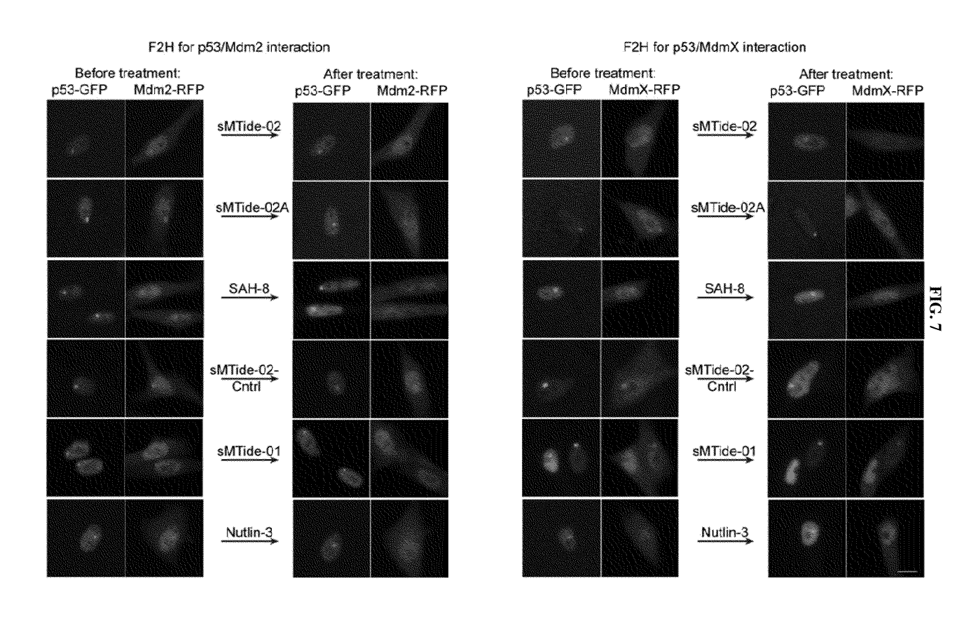

To further investigate the mechanism of action of the stapled peptides in live cells, a fully reversible cellular protein-protein interaction assay was used. The Fluorescent 2-Hybrid (F2H) assay (ChromoTek GmbH) is a microscopy-assisted method developed to analyze the disruption of the p53 interaction with either Mdm2 or Mdm4 within the nucleus of BHK cells (FIGS. 2b and 2c). sMTide-02 and sMTide-02A were both shown to dissociate Mdm2 from p53 with the former showing greater potency against Mdm4. Interestingly, the SAH-8 peptide showed poor activity in this assay and limited ability to inhibit either Mdm2/4. The selectivity of nutlin towards Mdm2 was exquisitely demonstrated by the F2H assay. Live cell observations revealed, that both the stapled peptides and nutlin dissociated Mdm2/Mdm4 from p53 within 60 minutes (FIG. 7).

The low activity of SAH-8 in the protein interaction assay and the T22 assay prompted the examination of the precise conditions used to measure the biological activity of stapled peptides. In previous studies, serum was removed from the media prior to addition of SAH-8 to cells, while the inventors' studies were conducted in the presence of fetal calf serum. The effect of serum on the activity of the stapled peptides was dramatic, with sMTide-02A, sMTide-02 and SAH-8 showing significantly improved potency in the absence of serum (FIG. 2d). However, the F2H assay showed a much smaller difference in the ability of the compounds to disrupt the complexes of Mdm2/4 with p53 in the presence or absence of serum, suggesting that serum removal sensitizes the p53 pathway to stimuli rather than limit peptide entry into cells.

To further probe the mechanism of action of the stapled peptides, HCT-116 p53+/+ cells were pre-treated with the PGP inhibitor PSC-883, a non-immunosuppressive cyclosporine A analogue. The titration of sMTide-02 with PSC-883 in the reporter assay significantly improved the sensitivity of the p53 response (FIG. 2e), but the titration with nutlin yielded no such improvement (FIG. 2f). Interestingly, the toxicity observed when nutlin is titrated alone (the decrease in p53 dependent reporter gene product due to cell death) occurred at lower concentrations in the presence of PSC-883. Inhibition of the PGP efflux pump therefore seemed to be potentiating the presumed p53 independent cellular toxicity of nutlin, whilst having no effect on p53 induction, as this is already efficiently activated.

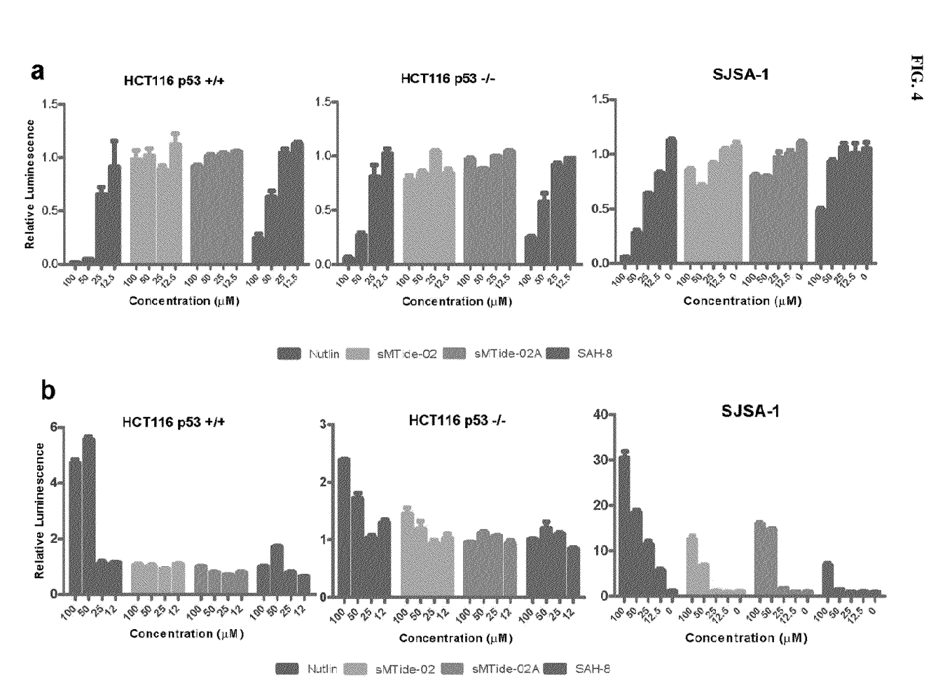

The effects of nutlin and the improved specificity of sMTide-02/02A were further studied in isogenic cell lines, that were either wild-type or null for p53 (FIGS. 4a and 3b). sMTide-02 and sMTide-02A caused no significant decrease in the viability of either HCT-116 p53+/+ or HCT-116 p53−/− cells, and induced negligible caspase 3/7 activity. Nutlin, and surprisingly SAH-8, exhibited distinctly different characteristics to the sMTide-02/02A peptides, with both compounds decreasing cell viability at high concentrations in both cell lines. Interestingly, nutlin induced caspase 3/7 activity in the two cell lines tested, with higher fold levels observed in HCT-116 p53+/+ cells.

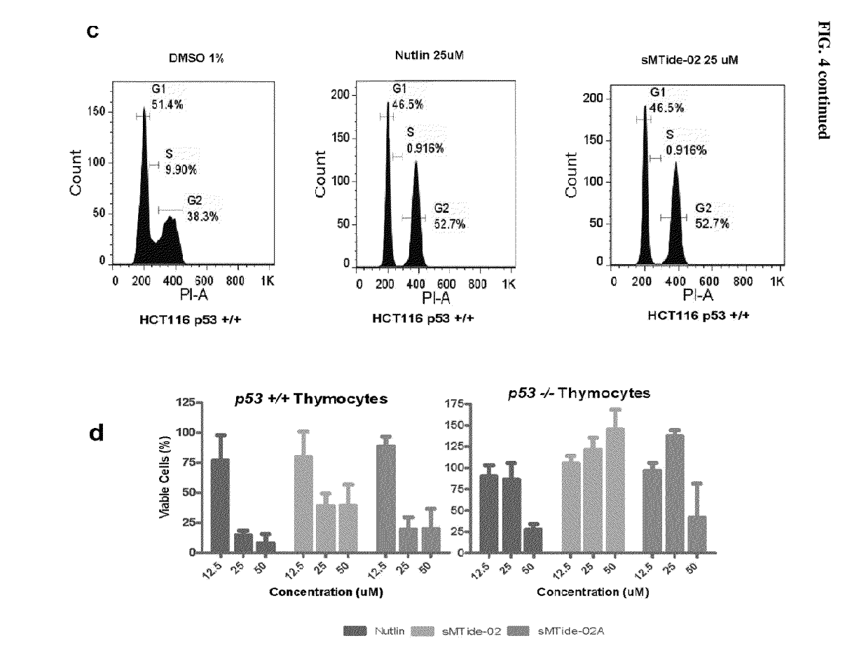

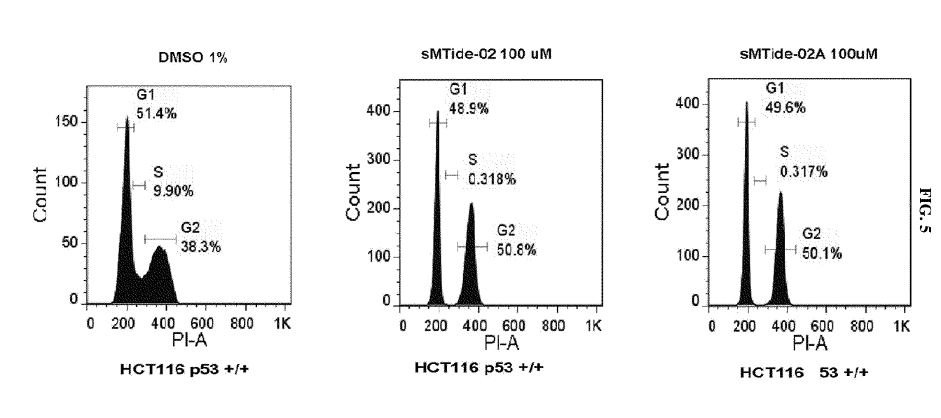

Analysis of the cell cycle distribution of cells possessing wild type p53 indicated that the sMTide-02/02A peptides caused G₂ arrest at both low (FIG. 4c) and high concentrations (FIG. 5), whilst nutlin at low concentrations also caused G₂ arrest (FIG. 4c) but at higher concentrations caused substantial cell death. SAH-8 stabilizes p53 at much higher concentrations than nutlin and sMTide-02/02A. This correlates with the observed decreases in cellular viability for the p53 wild type and null cells (FIG. 4a). These results indicate that (a) sMTide-02 and sMTide-02A induce a specific p53 response when they inhibit Mdm2/4 repression in HCT-116 p53+/+ cells but do not cause the cells to undergo p53 dependent apoptosis; (b) nutlin, at high concentrations, induces apoptosis in a p53 independent manner indicating an off target effect; (c) SAH-8 causes substantial cell death, irrespective of the presence of the wild type p53 gene.

To ensure that the sMTide-02/02A peptides could still induce apoptosis, they were titrated into the SJSA-1 cell line, which are sensitive to p53-dependent cell death. Both peptides induced caspase 3/7 activity at substantially higher concentrations than nutlin, which suggests that the off-target effect of nutlin plays a role in the efficient induction of apoptosis in SJSA-1 cells (FIGS. 4a and 4b). Primary thymocytes are also known to be sensitive to p53 dependent radiation induced apoptosis. Thymocytes were isolated from p53 wild-type and p53 deficient mice and then treated with nutlin or the sMTide-02/02A peptides. In this assay, the two peptides induced cell death in a p53-dependent manner. Notably, high doses of nutlin, and to a certain extent sMTide-02A, caused apoptosis in p53 null thymocytes, but sMTide-02 did not. This indicates an extraordinary degree of p53 dependent specificity (FIG. 4d), confirming that in this cell type, at least, p53 activation is sufficient to induce cell death.

These results suggest that when p53 is activated by inhibition of Mdm2 repression, an additional cellular signal is often required to induce efficient apoptosis of cells. With Nutlin, the signal that causes the larger increase of caspase 3/7 activity in cells possessing wild-type p53 may reside in the non-specific effects observed in the cells null for p53. Indeed, nutlin has been reported to interact with the BCL protein family. Such a mechanism implies that, for p53 reactivation to be a suitable therapy for cancer, an additional treatment (e.g. inhibition of proteins like MCL-1, BCL-2, ATM or MET) may be needed. Compared to sMTide-02/02A, SAH-8 leads to cell death in both isogenic HCT-116 cell lines, demonstrating the difference in origin of their respective peptide sequences. The p53 sequence, from which SAH-8 is derived, is also known to interact with other proteins (e.g. p300, TAFIIb) including Mdm2/4, which may explain the p53 independent cell death phenotype in p53 null cells and its toxicity to p53 wild-type cells. In contrast, the phage derived MTide sequence was selected to interact specifically with Mdm2/4 and also only encompasses the length of sequence required for binding to Mdm2/4.

The data presented validates stapled peptides as a new class of macrocyclic compounds, which are capable of interacting with intracellular targets with high affinity. The phage derived sMTide-02/02A compounds are more specific and potent in their mode of biological action than SAH-8, an existing peptide. These properties make the sMTide-02/02A peptides highly suitable for validating drug targets and even in parsing well understood small molecule therapies into specific and non-specific contributions. sMTide-02/02A are also suitable candidates for dual therapy treatments, in conjunction with, for example, an apoptosis promoting compound, and may be very useful in cyclotherapy approaches, where cellular arrest with low toxicity is required.

### Methods Section

Peptide Synthesis

The linear (MTide) peptides and stapled peptides (sMTide) was synthesized by Anaspec (San Diego, Calif.) by replacing the fourth (i) and eleventh (i+7) residues of the linear peptide with the olefin-bearing unnatural amino acids (R)-2-(7′octenyl) alanine and (S)-2-(4′-pentenyl) alanine respectively and stapled via olefin metathesis using the Grubbs catalyst. The staples peptide were purified using HPLC to >90% purity. Linear variant peptides were also synthesized by Anaspec (San Diego, Calif.) and purified using HPLC to >90% purity. All peptides were amidated at their C-terminus and acetylated at their N-terminus. The linear N-terminal FAM labelled peptide with the sequence RFMDYWEGL was synthesized by Mimotopes (Clayton, Australia) with the C-terminal amidated and was purified using HPLC to >90% purity.

Mdm2 and Mdm4 Purification

Mdm2 (1-125) and Mdm4 (1-125) were ligated into the GST fusion expression vector pGEX-6P-1 (GE Lifesciences) via a BAMH1 and NDE1 double digest. BL21 DE3 competent bacteria were then transformed with the GST tagged (1-125) Mdm2 and Mdm4 fusion constructs. The cells expressing the GST fusion constructs were grown in LB medium at 37° C. to an OD600 of ˜0.6 and induction was started with 1 mM IPTG and carried out overnight at room temperature. Cells were harvested by centrifugation and the cell pellets were re-suspended in 50 mM Tris pH 8.0, 10% sucrose and were then sonicated. The sonicated sample was centrifuged for 60 mins at 17,000 g at 4° C. The supernatant was applied to a 5 ml FF GST column (Amersham) pre-equilibrated in wash buffer (Phosphate Buffered Saline, 2.7 mM KCL and 137 mM NaCL, pH 7.4) with 1 mM DTT. The column was then further washed by 6 volumes of wash buffer. Mdm2 and Mdm4 were then purified from the column by cleavage with Precission (GE Lifesciences) protease. 10 units of precission protease, in one column volume of PBS with 1 mM DTT buffer, were injected onto the column. The cleavage reaction was allowed to proceed overnight at 4° C. The cleaved protein was then eluted of the column with wash buffer. Protein fractions were analyzed with SDS page gel and concentrated using a Centricon (3.5 kDa MWCO) concentrator, Millipore. Mdm2 and Mdm4 protein samples were then dialyzed into a buffer solution containing 20 mM Bis-Tris, pH 6.5, 0.05M NaCl with 1 mM DTT and loaded onto a monoS column pre-equilibrated in buffer A (20 mM Bis-Tris, pH 6.5, 1 mM DTT). The column was then washed in 6 column volumes of buffer A and bound protein was eluted with a linear gradient of 1M NaCL over 25 column volumes. Protein fractions were analyzed with SDS page gel and concentrated using a Centricon (3.5 kDa MWCO) concentrator from Millipore. The cleaved Mdm2 (1-125) and Mdm4 (1-125) constructs were purified to ˜90% purity. Protein concentration was determined using A280 with extinction coefficients of 10430 M⁻¹ cm⁻¹ and 7575 M⁻¹ cm⁻¹ for Mdm2 (1-125) and Mdm4 (1-125) respectively.

Computational Modeling

A computer model of the structure of the high affinity peptide MTide-01 (TSFAEYWNLLS) complexed to Mdm2 (PDB entry 3EQS) with a staple across positions 4 and 11 (I, I+7) was constructed. This was then mutated in silico at position 8 and the resulting models subject to detailed optimization.

Fluorescence Anisotropy Assays and K_{d} Determination

Purified Mdm2 (1-125) and Mdm4 (1-125) protein was titrated against 50 nM carboxyfluorescein (FAM) labelled 12-1 peptide (FAM-RFMDYWEGL-NH₂). Dissociation constants for titrations of Mdm2 and Mdm4 against FAM labelled 12-1 peptide were determined by fitting the experimental data to a 1:1 binding model equation shown below:

$$r = {r}_{o} + ( {r}_{b} - {r}_{o} ) \times \frac{( {K}_{d} + {[ L ]}_{t} + {[ P ]}_{t} ) - \sqrt{{{K}_{d} + {[ L ]}_{t} + {[ P ]}_{t} )}^{2} - {{4 [ L ]}_{t} [ P ]}_{t}}}{{2 [ L ]}_{t}}$$

where [P] is the protein concentration (Mdm2/Mdm4), [L] is the labelled peptide concentration, r is the anisotropy measured, r0 is the anisotropy of the free peptide, r_{b} is the anisotropy of the Mdm2/4-FAM-labelled peptide complex, K_{d} is the dissociation constant, [L]ₜ is the total FAM labelled peptide concentration, and [P]ₜ is the total Mdm2/4 concentration. The determined apparent K_{d} values (shown in the table below) were used in determining the apparent K_{d} values in subsequent competition assays, for both MDMX and MDM2, against the respective competing ligands:

| Peptide | Mdm2 K_{d} | Mdm4 K_{d} |
|---|---|---|
| (FAM)-RFMDYWEGL-NH₂ | 36.6 nM | 4.2 nM |

Apparent K_{d} values were determined for a variety of molecules via competitive fluorescence anisotropy experiments. Titrations were carried out with the respective concentrations of Mdm2 and Mdm4 held constant at 250 nM and 75 nM, respectively and the labelled peptide at 50 nM. The competing molecules were then titrated against complex of the FAM labelled peptide and protein. Apparent K_{d} values were determined by fitting the experimental data to the equations shown below:

$$r = {r}_{o} + ( {r}_{b} + {r}_{o} ) \times \frac{2 \sqrt{( {d}^{2} - 3 e )} cos ( θ / 3 ) - 9}{3 {K}_{d 1} + 2 \sqrt{( {d}^{2} - 3 e )} cos ( θ / 3 ) - d}$$

$$d = {K}_{d 1} + {K}_{d 2} + {[ L ]}_{st} + {[ L ]}_{t} - {[ P ]}_{t}$$

$$e = ( {[ L ]}_{t} - {[ P ]}_{t} ) {K}_{d 1} + ( {[ L ]}_{st} - {[ P ]}_{t} ) {K}_{d 2} + {K}_{d 1} {K}_{d 2}$$

$$f = - {K}_{d 1} {{K}_{d 2} [ P ]}_{t}$$

$$θ = ar cos [ \frac{- 2 {d}^{3} + 9 de - 27 f}{2 \sqrt{{( {d}^{2} - 3 e )}^{3}}} ]$$

[L]ₛₜ and [L]ₜ denote labelled ligand and total unlabelled ligand input concentrations, respectively. K_{d2} is the dissociation constant of the interaction between the unlabelled ligand and the protein. In all competitive types of experiments, it is assumed that [P]ₜ>[L]ₛₜ, otherwise considerable amounts of free labelled ligand would always be present and would interfere with measurements. K_{d1} is the apparent K_{d} for the labelled peptide used in the respective experiment, which has been experimentally determined as described in the previous paragraph. The FAM-labelled peptide were dissolved in DMSO at 1 mM and diluted into experimental buffer. Readings were carried out with a Envision Multilabel Reader (PerkinElmer). Experiments were carried out in PBS (2.7 mM KCl, 137 mM NaCl, 10 mM Na₂HPO₄ and 2 mM KH₂PO₄ (pH 7.4)) and 0.1% Tween 20 buffer. All titrations were carried out in triplicate. Curve-fitting was carried out using Prism 4.0 (GraphPad).

F2H Co-Localization Assay

The F2H assay is an intracellular, fully reversible protein-protein interaction assay. This microscopy-assisted assay consists of two components, a bait and a prey protein. The bait is a fusion of p53 (1-81) with a lac repressor binding domain (LacI) and GFP, whilst the prey is a fusion of either Mdm2 (7-134) or Mdm4 (1-129) with RFP. Upon expression in a transgenic BHK cell line containing lac operator repeats, the bait protein is captured at these repeats and forms a bright green spot in the nucleus. The prey protein interacts with the bait protein and localizes to the same spot in the cells. Compounds which inhibit the target interaction can then be titrated onto the cells and the declined percentage of co-localization is measured using imaging techniques. For testing the stapled peptides, BHK cells were co-transfected with the bait p53 and prey Mdm2/4 plasmids overnight in 96 multiwell plates (μClear Greiner Bio-One, Germany). The Lipofectamine 2000 (Life Technologies) reverse transfection protocol was applied according to manufacturer's instructions with 0.2 μg DNA and 0.4 μl Lipofectamine 2000 per well. Cells were incubated with a 2-fold dilution series of the relevant compounds from 50 to 1.5 μM in media with or without 10% FCS for 8 hours. Interaction (%) was determined as the ratio of cells showing co-localization of fluorescent signals at the nuclear spot to the total number of evaluated cells using an INCell Analyzer 1000 with a 20× objective (GE Healthcare). At least 100 co-transfected cells were analyzed per well. 2-fold titrations were carried out independently three times.

T22 p53 β-Galactosidase Based Reporter Assay

T22 cells, which were stably transfected with a p53 responsive 3-galactosidase reporter, were seeded into 96-well plate at a cell density of 8000 cells per well. Cells were also maintained in Dulbecco's Minimal Eagle Medium (DMEM) with 10% fetal bovine serum (FBS) and penicillin/streptomycin. The cells were incubated for 24 hours and then treated with compounds/peptide for 18 hours in DMEM with 10% FBS. β-galactosidase activity was detected using the FluoReporter LacZ/Galactosidase Quantitation kit (Invitrogen) as per manufacturer's instructions. Measurements were carried out using a Safire II multiplate reader (TECAN). Experiments were carried out independently twice.

Viability and Caspase-3/7 Cell Assays

Either 10,000 HCT p53+/+ or HCT p53−/− cells were seeded into 96 well plates and incubated overnight. Cells were also maintained in Dulbecco's Minimal Eagle Medium (DMEM) with 10% fetal bovine serum (FBS) and penicillin/streptomycin. The cells were then treated with the linear and stapled peptides the following morning for 24 hours in DMEM with 10% FBS. Cell viability was assayed by addition of CellTiter-Glo chemiluminescence reagent as according to the manufacturer's instructions and luminescence was measured using an Envision multi-plate reader (Perkin Elmer). Data was normalized to vehicle-treated controls. Equivalent experiments were performed and caspase-3/7 activity was assayed by addition of Caspase-Glo 3/7 chemiluminescence reagent according to the manufacturer's protocol (Promega) and luminescence was measured using an Envision multi-plate reader (Perkin Elmer). Data was shown as fold activation with respect to vehicle-treated controls. Experiments were carried out in triplicate and repeated independently twice.

HCT-116 Western Blot Analysis

HCT p53+/+ or HCT p53−/− cells were seeded into 6-well plates at a cell density of 350 000 cells per well and incubated overnight. Cells were also maintained in Macoy's media with 10% fetal bovine serum (FBS) and penicillin/streptomycin. Cells were treated with various compounds/vehicle controls at the time points and concentrations indicated also in acoy's media with 10% FBS. Cells were rinsed with PBS and then harvested in 200 μl of 1× NuPAGE LDS sample buffer supplied by Invitrogen (NP0008). Samples were then sonicated, heated to 90° C. for 5 mins, sonicated twice for 10 s and centrifuged at 13, 000 rpm for 5 minutes. Protein concentrations were measured by BCA assay (Pierce). Western blots were then performed using antibodies against actin (AC-15, Sigma) as a loading control, p21 (118 mouse monoclonal), Mdm2 (4B2 mouse monoclonal) and p53 (DO-1 mouse monoclonal).

Analysis of Cell Viability in Mouse Thymocytes

Thymocytes were isolated from wild-type and p53^{+/-}mice (age 4-9 weeks) and kept in a PBS/FCS (2%) solution (ref). Cells were plated at a density of 1×10⁶ per well in 24-well plates in medium [DMEM/Hepes (25 mM, pH 7.2), 5% FCS, penicillin/streptomycin, glutamine] and incubated at 37° C. with 2% C0₂. Cells were treated with nutlin, sMTide-02 or sMTide02A at concentrations of 12.5, 25 and 5 μM for a period of 24 hours. Cell were then analysed for viability using this procedure, cells were washed with cold PBS, and stained with FITC-labeled annexin V antibody (PharMingen) and PI (Sigma). The relative amounts of apoptotic cells were determined by binding of annexin V and subsequent fluorescence-activated cell sorter analysis. All values were normalized to the number of cells remaining viable in vehicle (1% DMSO) treated cultures derived from the same animal stained simultaneously. Data are representatives of n=4 independent experiments.

Cell Cycle Analysis of HCT-116 p53−/− and +/+ Cells

HCT-116 p53−/− and +/+ cells were cultured in McCoy's with 10% FCS and penicillin/streptomycin. Three hundred fifty thousand cells were then seeded per well in 6-well plates containing incubated overnight. After overnight incubation, the cells were treated with nutlin, the sMTide-02/02A peptides or SAH-8 at concentration of 25 and a 100 μM for 24 hours. A 1% DMSO vehicle control treatment was also carried out. Cells were washed with PBS and then detached from the plate surface by trypsinisation. Cells were then fixed in 65% ethanol/PBS and incubated at 4° C. for 2 h. Cells were spun down and resuspended with PI-staining solution containing RNase A. Cells were analyzed using an LSR II (Becton Dickinson).

1. A peptide comprising or consisting of the amino acid sequence of:
TSFXaa₁EYWXaa₃LLXaa₂
wherein Xaa₁ and Xaa₂ are any type of amino acid; and
wherein Xaa₃ is N or A and wherein in case Xaa₃ is N, Xaa₁ is not A and/or Xaa₂ is not S.

2. A peptide comprising or consisting of the amino acid sequence of:
TSFXaa₁EYWXaa₃LLXaa₂
wherein Xaa₁, Xaa₂ and Xaa₃ is independently any type of amino acid; and
wherein the peptide is a crosslinked peptide with a cross-linker to connect a first amino acid Xaa₁ to a second amino acid Xaa₂.

3. A peptide comprising or consisting of the amino acid sequence of:
TSFXaa₁EYWXaa₃LLXaa₂Xaa₄
wherein Xaa₁, Xaa₂ and Xaa₃ is independently any type of amino acid; and
wherein Xaa₄ is any type of amino acid other than P.

4. The peptide of claim 3, wherein Xaa₃ is N or A and wherein in case Xaa₃ is N, Xaa₁ is not A and/or Xaa₂ is not S.

5. The peptide of claim 3 or 4, wherein the peptide is a crosslinked peptide with a cross-linker to connect a first amino acid Xaa₃ to a second amino acid Xaa₂; and wherein Xaa₁, Xaa₂, Xaa₃ and Xaa₄ is independently any type of amino acid.

6. The peptide of any one of claims 1 to 5, wherein the peptide has a length of between about 4 to 15 amino acids.

7. The peptide of any one of claims 1 to 6, wherein the peptide which has undergone a post-translational modification selected from the group consisting of addition of one or more phosphoryl groups.

8. The peptide of any one of claims 1 to 7, wherein the peptide is modified to include one or more ligands selected from the group consisting of: hydroxyl, phosphate, amine, amide, sulphate, sulphide, a biotin moiety, a carbohydrate moiety, a fatty acid-derived acid group, a fluorescent moiety, a chromophore moiety, a radioisotope, a PEG linker, an affinity label, a targeting moiety, an antibody, a cell penetrating peptide and a combination of the aforementioned ligands.

9. The peptide of any one of claims 1 to 8, wherein the nitrogen of the backbone of the peptide is methylated.

10. The peptide of any of claims 1 to 9, wherein the peptide is fused to a heterologous polypeptide sequence.

11. The peptide according to any one of the preceding claims, wherein W at position 7 of the peptide is modified by addition of one or more halogen independently selected from the group consisting of F, Cl, Br, and I.

12. The peptide of claim 11, wherein the peptide comprises 1, 2, 3, 4, or 5 halogens.

13. The peptide of claim 11, wherein W at position 7 is modified by addition of a halogen at position C₆ of W and/or wherein W is independently an L or D optical isomer.

14. The peptide of any one of claims 8 to 10, wherein the halogen is Cl.

15. The peptide of claim 2, wherein the peptide comprises the formula: 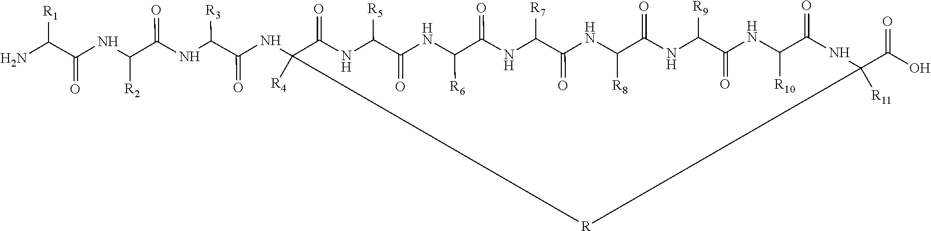
wherein:
R₁ is —C(OH)CH₃ [T];
R₂ is —CH₂OH [S];
R₃ is benzyl [F];
R₄ and R₁₁ are independently H or a C₁ to C₁₀ alkyl, alkenyl, alkynyl, arylalkyl, cycloalkylalkyl, heteroarylalkyl, or heterocyclylalkyl;
R₅ is —(CH₂)₂C(O)OH [E];
R₆ is —CH₂-Phenyl-OH [Y];
R₇ is the side chain of Trp, wherein C₆ of Trp is substituted with a hydrogen or a halogen and/or wherein Trp is independently an L or D optical isomer,
R₈ is the side chain of any amino acid;
R₉ and R₁₀ are —CH₂CH(CH₃)₂ [L];
R is alkyl, alkenyl, alkynyl; [R′—K—R″]ₙ; each of which is substituted with 0-6 R₁₂;
R′ and R″ are independently alkylene, alkenylene or alkynylene;
each R₁₂ is independently halo, alkyl, OR₁₃, N(R₁₃)₂, SR₁₃, SOR₁₃, SO₂R₁₃, CO₂R₁₃, R₁₃, a fluorescent moiety, or a radioisotope;
K is independently O, S, SO, SO₂, CO, CO₂, CONR₁₃ or;
each R₁₃ is independently H, alkyl, or a therapeutic agent;
n is an integer from 1-4.

16. The peptide according to claim 15, wherein R₄ and R₁₁ are independently H or C₁-C₆ alkyl.

17. The peptide of claim 15, wherein R is C₈ alkyl.

18. The peptide of claim 15, wherein R is C₁₁ alkyl.

19. The peptide of claim 15, wherein R is alkenyl.

20. The peptide of claim 15, wherein R is C₈ alkenyl.

21. The peptide of claim 15, wherein R is C₁₁ alkenyl.

22. The peptide of claim 15, wherein R₁, R₂, R₃, R₅, R₆, R₇, R₈, R₉, R₁₀ are as defined in claim 13; wherein R₄ and R₁₁ are H; and wherein R is C₁₁ alkenyl.

23. The peptide of claim 15, wherein R is a linear chain alkyl, alkenyl or alkynyl.

24. The peptide of claim 5, wherein the peptide comprises the formula: 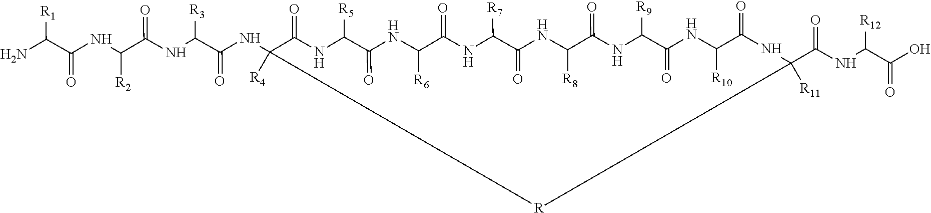
wherein:
R₁ is —C(OH)CH₃ [T];
R₂ is —CH₂OH [S];
R₃ is benzyl [F];
R₄ and R₁₁ are independently H or a C₁ to C₁₀ alkyl, alkenyl, alkynyl, arylalkyl, cycloalkylalkyl, heteroarylalkyl, or heterocyclylalkyl;
R₅ is —(CH₂)₂C(O)OH [E];
R₆ is —CH₂-Phenyl-OH [Y];
R₇ is the side chain of Trp, wherein C₆ of Trp is substituted with a hydrogen or a halogen and/or wherein Trp is independently an L or D optical isomer,
R₈ and R₁₂ are independently the side chain of any amino acid;
R₉ and R₁₀ are —CH₂CH(CH₃)₂ [L];
R is alkyl, alkenyl, alkynyl; [R′—K—R″]ₙ; each of which is substituted with 0-6 R₁₂;
R′ and R″ are independently alkylene, alkenylene or alkynylene;
each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope;
K is independently O, S, SO, SO₂, CO, CO₂, CONR₁₄ or;
each R₁₄ is independently H, alkyl, or a therapeutic agent;
n is an integer from 1-4.

25. The peptide according to claim 24, wherein R₄ and R₁₁ are independently H or C₁-C₆ alkyl.

26. The peptide of claim 24, wherein R is C₈ alkyl.

27. The peptide of claim 24, wherein R is C₁₁ alkyl.

28. The peptide of claim 24, wherein R is alkenyl.

29. The peptide of claim 24, wherein R is C₈ alkenyl.

30. The peptide of claim 24, wherein R is C₁₁ alkenyl.

31. The peptide of claim 24, wherein R₁, R₂, R₃, R₅, R₆, R₇, R₈, R₉, R₁₀ and R₁₂ are as defined in claim 14; wherein R₄ and R₁₁ are H; and wherein R is C₁₁ alkenyl

32. The peptide of claim 24, wherein R is a linear chain alkyl, alkenyl or alkynyl.

33. The peptide of claim 1, wherein Xaa₃ is any type of amino acid other than A and wherein in case Xaa₃ is N, Xaa₁ is not A and/or Xaa₂ is not S.

34. An isolated nucleic acid molecule encoding a peptide according to claim 1, 3, 4 or 6.

35. A vector comprising an isolated nucleic acid molecule according to claim 34.

36. A host cell comprising a nucleic acid molecule of claim 34 or a vector of claim 35.

37. A pharmaceutical composition comprising a peptide according to any one of claims 1 to 33, an isolated nucleic acid molecule according to claim 34, or a vector according to claim 35.

38. The pharmaceutical composition according to claim 37 further comprising one or more pharmaceutically acceptable excipients, vehicles or carriers.

39. The pharmaceutical composition according to claim 37 or 38, wherein the pharmaceutical composition comprises a further therapeutic compound.

40. The pharmaceutical composition of claim 39, wherein the further therapeutic compound is an apoptosis promoting compound.

41. The pharmaceutical composition of claim 40, wherein the apoptosis promoting compound is selected from the group consisting of Cyclin-dependent Kinase (CDK) inhibitors, Receptor Tyrosine Kinase (RTK) inhibitors, BCL (B-cell lymphoma) family BH3 (Bcl-2 homology domain 3)-mimetic inhibitors and Ataxia Telangiectasia Mutated (ATM) inhibitors.

42. The pharmaceutical composition of claim 41, wherein the CDK inhibitors comprise inhibitors selected from the group consisting of:
2-(R)-(1-Ethyl-2-hydroxyethylamino)-6-benzylamino-9-isopropylpurine (CYC202; Roscovitine; Seliciclib);
4-[[5-Amino-1-(2,6-difluorobenzoyl)-1H-1,2,4-triazol-3-yl]amino]benzenesulfonamide (JNJ-7706621);
N-(4-piperidinyl)-4-(2,6-dichlorobenzoylamino)-1H-pyrazole-3-carboxamide (AT-7519);
N-(5-(((5-(1,1-dimethylethyl)-2-oxazolyl)methyl)thio)-2-thiazolyl)-4-piperidinecarboxamide (SNS-032);
8,12-Epoxy-1H,8H-2,7b,12a-triazadibenzo(a,g)cyclonona(cde)triinden-1-one, 2,3,9,10,11,12-hexahydro-3-hydroxy-9-methoxy-8-methyl-10-(methylamino)-(UCN-01; 7-Hydroxystaurosporine; KRX-0601);
N,1,4,4-tetramethyl-8-((4-(4-methylpiperazin-1-yl)phenyl)amino)-4,5-dihydro-1H-pyrazolo[4,3-h]quinazoline-3-carboxamide (PHA-848125; milciclib);
2-(2-chlorophenyl)-5,7-dihydroxy-8-[(3S,4R)-3-hydroxy-1-methylpiperidin-4-yl]chromen-4-one hydrochloride (flavopiridol; alvocidib);
6-acetyl-8-cyclopentyl-5-methyl-2-((5-(piperazin-1-yl)pyridin-2-yl)amino)pyrido[2,3-d]pyrimidin-7(8H)-one hydrochloride (PD 0332991);
4-(1-isopropyl-2-methyl-1H-imidazol-5-yl)-N-(4-(methylsulfonyl)phenyl)pyrimidin-2-amine (AZD5438);
(S)-3-(((3-ethyl-5-(2-(2-hydroxyethyl)piperidin-1-yl)pyrazolo[1,5-a]pyrimidin-7-yl)amino)methyl)pyridine 1-oxide (Dinaciclib; SCH 727965);
N-(4-Piperidinyl)-4-(2,6-dichlorobenzoylamino)-1H-pyrazole-3-carboxamide hydrochloride (AT-7519); and pharmaceutically acceptable salts thereof.

43. The pharmaceutical composition of claim 41, wherein the RTK inhibitors comprise inhibitors selected from the group consisting of:
N-[3-chloro-4-[(3-fluorophenyl)methoxy]phenyl]-6-[5-[(2-methylsulfonylethylamino)methyl]-2-furyl]quinazolin-4-amine (lapatinib);
N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (foretinib);
N-(4-((6,7-Dimethoxyquinolin-4-yl)oxy)phenyl)-N-(4-fluorophenyl)cyclopropane-1,1-dicarboxamide (cabozantinib (XL184));
N-(4-((6,7-Dimethoxyquinolin-4-yl)oxy)phenyl)-N-(4-fluorophenyl)cyclopropane-1,1-dicarboxamide (cabozantinib (XL184));
3-[(1R)-1-(2,6-dichloro-3-fluorophenyl)ethoxy]-5-(1-piperidin-4-ylpyrazol-4-yl)pyridin-2-amine (crizotinib (Xalkori));
(3Z)—N-(3-Chlorophenyl)-3-({3,5-dimethyl-4-[(4-methylpiperazin-1-yl)carbonyl]-1H-pyrrol-2-yl}methylene)-N-methyl-2-oxo-2,3-dihydro-1H-indole-5-sulfonamide (SU11274);
(3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one hydrate (PHA-665752);
6-[[6-(1-Methylpyrazol-4-yl)-[1,2,4]triazolo[4,3-b]pyridazin-3-yl]sulfanyl]quinoline (SGX-523);
4-[1-(6-Quinolinylmethyl)-1H-1,2,3-triazolo[4,5-b]pyrazin-6-yl]-1H-pyrazole-1-ethanol methanesulfonate (1:1) (PF-04217903);
2-Fluoro-N-methyl-4-[7-[(quinolin-6-yl)methyl]imidazo[1,2-b]-[1,2,4]triazin-2-yl]benzamide (INCB28060);
N-[4-[(3-Chloro-4-fluorophenyl)amino]-7-[[(3S)-tetrahydro-3-furanyl]oxy]-6-quinazolinyl]-4(dimethylamino)-2-butenamide (afatinib);
3-(5,6-Dihydro-4H-pyrrolo[3,2,1-ij]quinolin-1-yl)-4-(1H-indol-3-yl)-pyrrolidine-2,5-dione (ARQ-197 (Tivantinib));
N-[(2R)-1,4-dioxan-2-ylmethyl]-N-methyl-N-[3-(1-methyl-1H-pyrazol-4-yl)-5-oxo-5H-benzo[4,5]cyclohepta[1,2-b]pyridin-7-yl]sulfuric diamide (MK-2461);
N-[4-(3-Amino-1H-indazol-4-yl)phenyl]-N-(2-fluoro-5-methylphenyl)urea (Linifanib (ABT 869));
4-[[(3S)-3-Dimethylaminopyrrolidin-1-yl]methyl]-N-[4-methyl-3-[(4-pyrimidin-5-ylpyrimidin-2-yl)amino]phenyl]-3-(trifluoromethyl)benzamide (Bafetinib (INNO-406)); and pharmaceutical salts thereof.

44. The pharmaceutical composition of claim 41, wherein the BCL family BH3-mimetic inhibitors comprise inhibitors selected from the group consisting of:
4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (ABT 263; Navitoclax);
2-[2-[(3,5-Dimethyl-1H-pyrrol-2-yl)methylene]-3-methoxy-2H-pyrrol-5-yl]-1H-indole methanesulfonate (Obatoclax mesylate (GX15-070));
4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737); and pharmaceutically acceptable salts thereof.

45. The pharmaceutical composition of claim 41, wherein the ATM inhibitors comprise inhibitors selected from the group consisting of:
2-Morpholin-4-yl-6-thianthren-1-yl-pyran-4-one (KU-55933);
(2R,6S)-2,6-Dimethyl-N-[5-[6-(4-morpholinyl)-4-oxo-4H-pyran-2-yl]-9H-thioxanthen-2-yl]-4-morpholineacetamide (KU-60019);
1-(6,7-Dimethoxy-4-quinazolinyl)-3-(2-pyridinyl)-1H-1,2,4-triazol-5-amine (CP466722);
α-Phenyl-N-[2,2,2-trichloro-1-[[[(4-fluoro-3-nitrophenyl)amino]thioxomethyl]amino]ethyl]benzene acetamide (CGK 733)
and pharmaceutically acceptable salts thereof.

46. Use of the peptide according to any one of claims 1 to 33 in the manufacture of a medicament for treating or preventing cancer.

47. The use according to claim 46, wherein cancer comprises a tumor comprising a non-mutant p53 sequence.

48. The use according to claim 47 or 48, wherein cancer is selected from a group comprising or consisting of gastric cancer, colon cancer, lung cancer, breast cancer, bladder cancer, neuroblastoma, melanoma, and leukemia.

49. Method of treating or preventing cancer in a patient comprising administering a pharmaceutically effective amount of the peptide of any one of claims 1 to 33 or the isolated nucleic acid molecule according to claim 34, or the vector according to claim 35.

50. The method according to claim 49 wherein the method comprises the administration of one or more further therapeutic agents to the patient, wherein administration is simultaneous, sequential or separate.

51. The method of claim 49 or 50, wherein administration of the peptide induces a reversible cell cycle arrest in non-cancerous proliferating cells.

52. The method of any one of claims 49 to 51, wherein the patient suffering or suspected to suffering from cancer comprises a tumor with p53 deficient tumor cells or p53 genes comprising a mutation which causes the cancer.
